(19) United States
(12) Patent Application Publication
(10) Pub. No.: US 2021/0253623 A1
(43) Pub. Date: Aug. 19, 2021

(54) Nucleic acid unit and polymeric nucleic acid and application thereof

(71) Applicant: GUANGZHOU RIBOBIO CO., LTD., Guangzhou, Guangdong (CN)

(72) Inventors: Biliang ZHANG, Boston, MA (US); Xiuqun YANG, Guangzhou, Guangdong (CN); Dmitry SAMARSKY, Boston, MA (US)

(21) Appl. No.: 17/042,946

(22) PCT Filed: Jul. 20, 2018
(86) PCT No.: PCT/CN2018/096397

§ 371 Date: Sep. 29, 2020

(51) Int. Cl. C07H 21/00; A61P 35/00

(52) U.S. Cl. CPC C07H 21/00; A61P 35/00

(57) ABSTRACT

Disclosed are a new-type nucleic acid unit for construction of a polymeric nucleic acid and the polymeric nucleic acid for interfering with target gene expression. In the present application, by design and construction of the new-type nucleic acid unit and the self-assembled polymeric nucleic acid thereof, multiple target interference may be realized, wherein the same may be used for inhibiting multiple gene expressions in a signaling pathway of disease occurrence or development, or simultaneously inhibiting multiple disease target genes expression, possessing broad application prospects in multiple subject fields such as biology and chemistry. The polymeric nucleic acid may target multiple sequences simultaneously, wherein the sequences may be located in one or more genes.

### CROSS-REFERENCE TO RELATED APPLICATIONS

This application is the national stage entry of PCT/CN2018/096397, filed on Jul. 20, 2018, which are incorporated by reference in their entirety herein.

### SEQUENCE LISTING

The instant application contains a Sequence Listing which has been submitted electronically in ASCII format and is hereby incorporated by reference in its entirety. Said ASCII copy is named “PN135790_Sequence_Listing.txt” and is 37.3 kilobytes in size, and contains 24 new sequences from SEQ ID NO:102 to SEQ ID NO:125 disclosed in page 21 of this file and Table 8, but not numbered. The original sequences of SEQ ID NO: 1-SEQ ID NO:101 are identical to the sequence listing filed in the corresponding international application No. PCT/CN2018/096397 filed on Jul. 20, 2018, except that the feature data for each sequence is added to complete these artificial sequences.

### TECHNICAL FIELD

The present disclosure belongs to the technical field of nucleic acids, and particularly relates to a nucleic acid unit in new-type structure and a self-assembled polymeric nucleic acid thereof, the polymeric nucleic acid may interfere with expressions of one or more target genes.

### BACKGROUND

A complementary oligonucleotide (CON) technology makes an oligonucleotide molecule synthesized artificially bind to a target molecule through sequence complementation, and change the biological characteristics of the target molecule. The CON technology includes two types of a RNA interference (RNAi) technology and an antisense oligonucleotide (ASO) technology, it is widely used in functional genomics research at present, and expected to become the third greatest treatment means in addition to small molecule compounds and biological agents. For example, mipomersen for treating homozygous familial hypercholesterolemia (FoFH), it is a synthetic phosphorothioate oligonucleotide developed by Genzyme, through complementary pairing with a coding region of an Apo B-100 protein mRNA, translation and synthesis of an Apo B-100 protein (main apolipoproteins of LDL and VLDL) are inhibited, thereby LDL-C, TC, and Non-HDL-C levels of a FoFH patient are effectively reduced. Although the CON technology achieves a certain success, this technology still needs to be improved. For example, a traditional RNAi reagent has the following disadvantages: 1) complicated synthetic steps and relatively high production cost; 2) high sensitivity to endonuclease and exonuclease, and low stability; 3) inhibition efficiency is not high enough, it may not be guaranteed that a single molecule may inhibit an expression of a target gene necessarily; 4) side effects caused by non-specific activity, the non-specific effect is mainly derived from a sense strand; and 5) it is difficult to be introduced into a cell, especially animals.

A nanotechnology researches properties and applications of a substance in diameter within a nano range of 1-100 nm. The characteristics, such as a small size effect, a surface effect and a high diffusivity, of the substance in nanostructure open up a new field for scientific research and technical application. The oligonucleotide is flexible in structure, a RNA structure for example, it may form a nanostructure by self-assembly. A nucleic acid nanotechnology represented by DNA and RNA is a new direction of development in the field of nanotechnologies.

### SUMMARY

A technical problem to be solved by the present disclosure is how to inhibit or reduce or interfere with an expression of a target gene.

In order to solve the above technical problem, the present disclosure firstly provides a polymeric nucleic acid molecule for inhibiting or reducing or interfering with the expression of the target gene.

The polymeric nucleic acid molecule for inhibiting or reducing or interfering with the expression of the target gene provided by the present disclosure is formed by n X-type nucleic acid molecules.

Each of the X-type nucleic acid molecules is formed by a targeting segment I, a targeting segment II and a linker segment III successively from a 5′-end. The targeting segment I of each of the X-type nucleic acid molecules is complementary-paired with the linker segment III of an adjacent X-type nucleic acid molecule thereof. Specifically, the n X-type nucleic acid molecules are respectively named as an X1 unit, an X2 unit, an X3 unit, and so on, an Xn-1 unit, and an Xn unit; the linker segment III of the X1 unit is complementary-paired (complete complementary) with the targeting segment I of the X2 unit, the linker segment III of the X2 unit is complementary-paired (complete complementary) with the targeting segment I of the X3 unit, and so on, the linker segment III of the Xn-1 unit is complementary-paired (complete complementary) with the targeting segment I of the Xn unit, and the linker segment III of the Xn unit is complementary-paired (complete complementary) with the targeting segment I of the X1 unit. The targeting segment I of each of the X-type nucleic acid molecules is not complementary to other X-type nucleic acid molecule sequences.

The targeting segment I and the targeting segment II of each of the polymeric nucleic acid molecules are complementary-paired with a target gene; each of the polymeric nucleic acid molecules is combined with a specific sequence of the target gene through the targeting segment I and the targeting segment II, so as to inhibit or reduce or interfere with the expression of the target gene. In certain specific cases, a region complementary to a target gene sequence may be prolonged to a partial sequence of the linker segment III. The targeting segment I and the targeting segment II in the same X-type nucleic acid molecule may be the same, or may be different. The targeting segment I or the targeting segment II in two different X-type nucleic acid molecules may be the same, or may be different.

A length of each of the X-type nucleic acid molecules is the same (a structure is the same too).

The n is an integer greater than or equal to 3.

In a specific embodiment of the present disclosure, the n X-type nucleic acid molecules are successively connected end to end (achieved by complementation of the linker segment III of the previous X-type nucleic acid molecule and the targeting segment I of the next adjacent X-type nucleic acid molecule), finally a polymeric nucleic acid molecule with a cyclic secondary structure is formed.

In the above polymeric nucleic acid molecule, the n X-type nucleic acid molecules may also form a polymeric nucleic acid molecule with a linear structure, the polymeric nucleic acid molecule with the linear structure further includes a H-type nucleic acid molecule, and the H-type nucleic acid molecule is formed by a H1-type nucleic acid molecule and a Hn-type nucleic acid molecule.

n X-type nucleic acid molecules are respectively named as an X1 unit, an X2 unit, an X3 unit, and so on, an Xn-1 unit, and an Xn unit; the linker segment III of the X1 unit is complementary-paired with the targeting segment I of the X2 unit, the linker segment III of the X2 unit is complementary-paired with the targeting segment I of the X3 unit, and so on, the linker segment III of the Xn-1 unit is complementary-paired with the targeting segment I of the Xn unit; the H1-type nucleic acid molecule is complementary-paired with the targeting segment I of the X1 unit; and the Hn-type nucleic acid molecule is complementary-paired with the linker segment III of the Xn unit.

Further, 5′-end or 3′-end of the H-type nucleic acid molecule further includes a Hy segment; and the H-type nucleic acid molecule is formed by an Hx segment and the Hy segment.

A complementary region of the H1-type nucleic acid molecule and the X1 unit may be extended to partial or all sequences of the targeting segment II of the X1 unit; and a complementary region of the Hn-type nucleic acid molecule and the Xn unit may be extended to partial or all sequences of the targeting segment II of the Xn unit.

The Hx segment of the H1-type nucleic acid molecule is complementary-paired with the targeting segment I of the X1 unit; the Hx segment of the Hn-type nucleic acid molecule is complementary-paired with the linker segment III of the Xn unit; and the Hy segment is complementary-paired with one, two or more continuous nucleic acid molecules of the targeting segment II of the X1 unit or the Xn unit from 5-end or 3′-end.

In the above polymeric nucleic acid molecules, the polymeric nucleic acid molecule with the cyclic structure further includes a C-type nucleic acid molecule; the C-type nucleic acid molecule is formed by n segments connected successively, the n segments are respectively reverse complementary sequences of the targeting segments II in the n X-type nucleic acid molecules. Specifically, the C-type nucleic acid molecule is formed by a capping-end segment 1, a capping-end segment 2, a capping-end segment 3, and so on, a capping-end segment Cn-1, and a capping-end segment n successively from a 3-end; and n X-type nucleic acid molecules are respectively named as an X1 unit, an X2 unit, an X3 unit, and so on, an Xn-1 unit, and an Xn unit;

the linker segment III of the X1 unit is complementary-paired with the targeting segment I of the X2 unit, the linker segment III of the X2 unit is complementary-paired with the targeting segment I of the X3 unit, and so on, the linker segment III of the Xn-1 unit is complementary-paired with the targeting segment I of the Xn unit, and the linker segment III of the Xn unit is complementary-paired with the targeting segment I of the X1 unit; the capping-end segment 1 is complementary-paired with the targeting segment II of the X1 unit, the capping-end segment 2 is complementary-paired with the targeting segment II of the X2 unit, and so on, the capping-end segment Cn-1 is complementary-paired with the targeting segment II of the Xn-1 unit, and the capping-end segment n is complementary-paired with the targeting segment II of the Xn unit.

In the above polymeric nucleic acid molecules, the nucleic acid molecule (the X-type nucleic acid molecule, the H-type nucleic acid molecule or the C-type nucleic acid molecule) may be a DNA or a RNA or an oligonucleotide formed by the DNA and the RNA.

Further, the nucleic acid molecule (the X-type nucleic acid molecule, the H-type nucleic acid molecule or the C-type nucleic acid molecule) is a single-stranded RNA molecule.

In the above polymeric nucleic acid molecules, a length of the X-type nucleic acid molecule is 15-50 nt, preferably 24-36 nt;

a length of the targeting segment I is 5-24 nt;

a length of the targeting segment II is 1-20 nt;

a length of the linker segment III is 5-24 nt;

a sum of the lengths of the targeting segment I and the targeting segment II is 14-16 nt at least; and

a length of the Hy segment is 2-6 nt or longer.

Further, the length of the X-type nucleic acid molecule is 24-36 nt.

In the above polymeric nucleic acid molecules, the polymeric nucleic acid molecule at least includes a modified nucleotide.

Further, the modification is phosphoric acid backbone modification, base modification and/or ribose modification. The ribose modification is that a ribose 2-site hydroxyl group is substituted by a halogen group or an O-alkyl group. The alkyl is a methyl, an ethyl, a propyl or a methylethyl.

Furthermore, the ribose 2-site hydroxyl groups of 5-9 continuous nucleotides, from the first nucleotide at the 3′-end, of the linker segment III of the X-type nucleic acid molecule are substituted by the halogen group or the O-alkyl group;

the ribose 2-site hydroxyl groups of 5-9 continuous nucleotides, from the first nucleotide at the 3′-end, of the Hx segment of the H-type nucleic acid molecule are substituted by the halogen group or the O-alkyl group;

the ribose 2-site hydroxyl groups of 8-30 continuous nucleotides, from the first nucleotide at the 3′-end, of the H-type nucleic acid molecule are substituted by the halogen group or the O-alkyl group; preferably, the ribose 2-site hydroxyl groups of 14-18 continuous nucleotides, from the first nucleotide at the 3′-end, of the H-type nucleic acid molecule are substituted by the halogen group or the O-alkyl group; and

the ribose 2-site hydroxyl groups of 2-6 continuous nucleotides, from the first nucleotide at the 5′-end, of each capping-end segment in the C-type nucleic acid molecule are substituted by the halogen group or the O-alkyl group.

In the above polymeric nucleic acid molecules, the n is 3 or 4 or 5 or 6 or 7 or 8.

Further, the n is 4 or 5 or 6.

In the above polymeric nucleic acid molecules, the number of the target genes is one or two or more, and the number of the target genes does not exceed n; and each X-type nucleic acid molecule corresponds to one target gene, or multiple X-type nucleic acid molecules correspond to different regions of the same target gene.

Further, the number of the target genes is 1 or 4 or 6.

The target gene is at least one of the following genes: PPIB, p65, BIRC5, CTNNB, COPS5, CLU, EIF4E, HIF1A, TP53, VEGFA and SOD1.

Furthermore, the polymeric nucleic acid molecule for interfering with an expression of the target gene PP1B is the following a1)-a5):

a1) is formed by single-stranded RNA molecules shown in SEQ ID NO:1, SEQ ID NO:2, SEQ ID NO:3 and SEQ ID NO:4;

a2) is formed by single-stranded RNA molecules shown in SEQ ID NO:1, SEQ ID NO:2, SEQ ID NO:3, SEQ ID NO:4, SEQ ID NO:20 and SEQ ID NO:21;

a3) is formed by single-stranded RNA molecules shown in SEQ ID NO:1, SEQ ID NO:2, SEQ ID NO:3, SEQ ID NO:4 and SEQ ID NO:8;

a4) is formed by single-stranded RNA molecules shown in SEQ ID NO:2, SEQ ID NO:3, SEQ ID NO:9, SEQ ID NO:10, SEQ ID NO:11 and SEQ ID NO:12;

a5) is formed by single-stranded RNA molecules shown in SEQ ID NO:2, SEQ ID NO:3, SEQ ID NO:6, SEQ ID NO:9, SEQ ID NO:10, SEQ ID NO:11, SEQ ID NO:13 and SEQ ID NO:14;

the polymeric nucleic acid molecule for interfering with an expression of the target gene P65 is the following b1)-b5):

b1) is formed by single-stranded RNA molecules shown in SEQ ID NO:15, SEQ ID NO:16, SEQ ID NO:17 and SEQ ID NO:18;

b2) is formed by single-stranded RNA molecules shown in SEQ ID NO:6, SEQ ID NO:7, SEQ ID NO:15, SEQ ID NO:16, SEQ ID NO:17 and SEQ ID NO:19;

b3) is formed by single-stranded RNA molecules shown in SEQ ID NO:15, SEQ ID NO:16, SEQ ID NO:17, SEQ ID NO:18 and SEQ ID NO:22;

b4) is formed by single-stranded RNA molecules shown in SEQ ID NO:15, SEQ ID NO:16, SEQ ID NO:17, SEQ ID NO:23, SEQ ID NO:24 and SEQ ID NO:25;

b5) is formed by single-stranded RNA molecules shown in SEQ ID NO:15, SEQ ID NO:16, SEQ ID NO:17, SEQ ID NO:20, SEQ ID NO:23, SEQ ID NO:24, SEQ ID NO:26 and SEQ ID N0:27;

the polymeric nucleic acid molecule for simultaneously interfering with expressions of the target genes BIRC5, CTNNB, COPS5 and CLU is formed by single-stranded RNA molecules shown in SEQ ID NO:28, SEQ ID NO:29, SEQ ID NO:30 and SEQ ID NO:31;

the polymeric nucleic acid molecule for simultaneously interfering expressions of the target genes BIRC5, CTNNB, COPS5, CLU, EIF4E and HIF1A is formed by single-stranded RNA molecules shown in SEQ ID NO:28, SEQ ID NO:29, SEQ ID NO:30, SEQ ID NO:32, SEQ ID NO:33 and SEQ ID NO:34;

the polymeric nucleic acid molecule for simultaneously interfering with expressions of the target genes SOD1, PPIB, P65 and VEGFA is the following c1)-c3):

c1) is formed by single-stranded RNA molecules shown in SEQ ID NO:35, SEQ ID NO:36, SEQ ID NO:37 and SEQ ID NO:38;

c2) is formed by single-stranded RNA molecules shown in SEQ ID NO:35, SEQ ID NO:36, SEQ ID NO:37, SEQ ID NO:38, SEQ ID NO:39 and SEQ ID NO:40;

c3) is formed by single-stranded RNA molecules shown in SEQ ID NO:35, SEQ ID NO:36, SEQ ID NO:37, SEQ ID NO:38 and SEQ ID NO:41;

the polymeric nucleic acid molecule for interfering with an expression of the target gene VEGFA is the following d1)-d11):

d1) is formed by single-stranded RNA molecules shown in SEQ ID NO:42, SEQ ID NO:43, SEQ ID NO:44, SEQ ID NO:45, sequence 46 and SEQ ID NO:47;

d2) is formed by single-stranded RNA molecules shown in SEQ ID NO:48, SEQ ID NO:49, SEQ ID NO:50, SEQ ID NO:51, SEQ ID NO:52 and SEQ ID NO:53;

d3) is formed by single-stranded RNA molecules shown in SEQ ID NO:54, SEQ ID NO:55, SEQ ID NO:56, SEQ ID NO:57, SEQ ID NO:58 and SEQ ID NO:59;

d4) is formed by single-stranded RNA molecules shown in SEQ ID NO:42, SEQ ID NO:43, SEQ ID NO:45, SEQ ID NO:46 and SEQ ID NO:47;

d5) is formed by single-stranded RNA molecules shown in SEQ ID NO:48, SEQ ID NO:49, SEQ ID NO:51, SEQ ID NO:52 and SEQ ID NO:53;

d6) is formed by single-stranded RNA molecules shown in SEQ ID NO:54, SEQ ID NO:55, SEQ ID NO:57, SEQ ID NO:58 and SEQ ID NO:59;

d7) is formed by single-stranded RNA molecules shown in SEQ ID NO:42, SEQ ID NO:45, SEQ ID NO:46 and SEQ ID NO:47;

d8) is formed by single-stranded RNA molecules shown in SEQ ID NO:48, SEQ ID NO:51, SEQ ID NO:52 and SEQ ID NO:53;

d9) is formed by single-stranded RNA molecules shown in SEQ ID NO:54, SEQ ID NO:57, SEQ ID NO:58 and SEQ ID NO:59;

d10) is formed by single-stranded RNA molecules shown in SEQ ID NO:60, SEQ ID NO:61, SEQ ID NO:62, SEQ ID NO:63, SEQ ID NO:64 and SEQ ID NO:65;

d11) is formed by single-stranded RNA molecules shown in SEQ ID NO:66, SEQ ID NO:67, SEQ ID NO:68, SEQ ID NO:69, SEQ ID NO:70 and SEQ ID NO:71;

the polymeric nucleic acid molecule for interfering with an expression of the target gene TP53 is the following e1)-e11):

e1) is formed by single-stranded RNA molecules shown in SEQ ID NO:72, SEQ ID NO:73, SEQ ID NO:74, SEQ ID NO:75, SEQ ID NO:76 and SEQ ID NO:77;

e2) is formed by single-stranded RNA molecules shown in SEQ ID NO:78, SEQ ID NO:79, SEQ ID NO:80, SEQ ID NO:81, SEQ ID NO:82 and SEQ ID NO:83;

e3) is formed by single-stranded RNA molecules shown in SEQ ID NO:84, SEQ ID NO:85, SEQ ID NO:86, SEQ ID NO:87, SEQ ID NO:88 and SEQ ID NO:89;

e4) is formed by single-stranded RNA molecules shown in SEQ ID NO:73, SEQ ID NO:74, SEQ ID NO:75, SEQ ID NO:76 and SEQ ID NO:77;

e5) is formed by single-stranded RNA molecules shown in SEQ ID NO:79, SEQ ID NO:80, SEQ ID NO:81, SEQ ID NO:82 and SEQ ID NO:83;

e6) is formed by single-stranded RNA molecules shown in SEQ ID NO:54, SEQ ID NO:55, SEQ ID NO:57, SEQ ID NO:58 and SEQ ID NO:59;

e7) is formed by single-stranded RNA molecules shown in SEQ ID NO:74, SEQ ID NO:75, SEQ ID NO:76 and SEQ ID NO:77;

e8) is formed by single-stranded RNA molecules shown in SEQ ID NO:80, SEQ ID NO:81, SEQ ID NO:82 and SEQ ID NO:83;

e9) is formed by single-stranded RNA molecules shown in SEQ ID NO:86, SEQ ID NO:87, SEQ ID NO:88 and SEQ ID NO:89;

e10) is formed by single-stranded RNA molecules shown in SEQ ID NO:90, SEQ ID NO:91, SEQ ID NO:92, SEQ ID NO:93, SEQ ID NO:94 and SEQ ID NO:95; and

e11) is formed by single-stranded RNA molecules shown in SEQ ID NO:96, SEQ ID NO:97, SEQ ID NO:98, SEQ ID NO:99, SEQ ID NO:100 and SEQ ID NO:101.

In order to solve the above technical problem, the present disclosure further provides a derivative of the above polymeric nucleic acid molecule.

The derivative of the above polymeric nucleic acid molecule provided by the present disclosure is any one of the following (m1)-(m5):

(m1) the above polymeric nucleic acid molecule is deleted or added one or more nucleotides, to obtain a derivative of the polymeric nucleic acid molecule having the same function as the polymeric nucleic acid molecule;

(m2) the above polymeric nucleic acid molecule is performed nucleotide substitution or modification, to obtain a derivative of the polymeric nucleic acid molecule having the same function as the polymeric nucleic acid molecule;

(m3) a backbone of the above polymeric nucleic acid molecule is transformed into a phosphorothioate backbone, to obtain a derivative of the polymeric nucleic acid molecule having the same function as the polymeric nucleic acid molecule;

(m4) a peptide nucleic acid, a locked nucleic acid or an unlocked nucleic acid coded by the above polymeric nucleic acid molecule is used, to obtain a derivative of the polymeric nucleic acid molecule having the same function as the polymeric nucleic acid molecule; and

(m5) one end or middle of the above polymeric nucleic acid molecule is linked with a signal molecule and/or an active molecule and/or a functional group, to obtain a derivative of the polymeric nucleic acid molecule having the same function as the polymeric nucleic acid molecule.

In order to solve the above technical problem, the present disclosure further provides a preparation method for the above polymeric nucleic acid molecule.

The preparation method for the above polymeric nucleic acid molecule provided by the present disclosure includes the following steps:

M1) the above X-type nucleic acid molecule and/or H-type nucleic acid molecule and/or C-type nucleic acid molecule is synthesized; and

M2) the X-type nucleic acid molecule and/or the H-type nucleic acid molecule and/or the C-type nucleic acid molecule is annealed, to obtain the polymeric nucleic acid.

In the above method, a reaction system for annealing is a system obtained by uniformly mixing each single-stranded RNA molecule in equal molar weight, RNA annealing buffer and water. A reaction condition for annealing is that a temperature is 90 DEG C in a polymerase chain reaction (PCR) meter, it is adequately denaturized in 2 min, and then the temperature in the PCR meter is reduced to 25 DEG C so that it is annealed.

While n is 4, the annealing system is as follows (a total volume is 100 μL): 20 μL of each single-stranded RNA molecule solution (the concentration is 100 μM), 15 μL of the RNA annealing buffer (5X), and 5 μL of the DEPC water.

While n is 5, the annealing system is as follows (the total volume is 150 μL): 20 μL of each single-stranded RNA molecule solution (the concentration is 150 μM), 30 μL of the RNA annealing buffer (5X), and 20 μL of the DEPC water.

While n is 6, the annealing system is as follows (the total volume is 200 μL): 20 μL of each single-stranded RNA molecule solution (the concentration is 200 μM), 40 μL of the RNA annealing buffer (5X), and 40 μL of the DEPC water.

In order to solve the above technical problem, the present disclosure further provides a new application of the above polymeric nucleic acid molecule or derivative.

The present disclosure provides the application of the above polymeric nucleic acid molecule or derivative in the following A1) or A2):

A1) a target gene expression level in a cell is controlled; and

A2) a product for preventing and/or relieving and/or treating a disease caused by the target gene expression is prepared.

In the above application, the control is inhibition or reduction or interference.

In the above application, the cell is a tumor cell.

In the above application, the target gene is a disease-related gene; the disease-related gene is a tumor-related gene specifically; and the tumor-related gene is specifically at least one of the following genes: PPIB, p65, BIRC5, CTNNB, COPS5, CLU, EIF4E, HIF1A, TP53, VEGFA and SOD1.

In order to solve the above technical problem, the present disclosure further provides a reagent or a kit or a drug for inhibiting or reducing or interfering with a target gene expression level in a cell.

The reagent or the kit or the drug for inhibiting or reducing or interfering with the target gene expression level in the cell provided by the present disclosure includes the above polymeric nucleic acid molecule or the above derivative.

In order to solve the above technical problem, the present disclosure finally provides a method for inhibiting or reducing or interfering with a target gene expression level in a cell.

The method for inhibiting or reducing or interfering the target gene expression level in the cell provided by the present disclosure includes the following steps: the above polymeric nucleic acid molecule or derivative is introduced into the cell, and the expression level of the target gene in the cell is inhibited or reduced.

In the above method, the introduction method is that the polymeric nucleic acid molecule, a transfection reagent and buffer are added to a cell culture medium after being uniformly mixed, to obtain a reaction system, herein the final concentration of the polymeric nucleic acid molecule in the reaction system is 1-300 nM.

In the above method, the cell is a tumor cell.

In the above method, the target gene is a disease-related gene; the disease-related gene is a tumor-related gene specifically; and the tumor-related gene is specifically at least one of the following genes: PPIB, p65, BIRC5, CTNNB, COPS5, CLU, EIF4E, HIF1A, TP53, VEGFA and SODS.

The advantages of the present disclosure are as follows:

1) RNAi efficiency is improved;

2) the structure is more stable, chemical stability is good, nuclease degradation resistance is enhanced, especially the stability in blood is enhanced, and a half-life period is prolonged;

3) the off-target rate is reduced;

4) a nano-particle structure may be formed, and the ability to introduce the cell is enhanced;

5) the nucleic acid molecule may be modular-designed; and

6) the polymeric nucleic acids in some structures do not require a Dicer enzyme to participate in the RNAi effect, therefore they are more resistant to chemical modification, and may be partially or completely modified.

The present disclosure is capable of enabling the CON technology to be combined with the nanotechnology, through constructing a nucleic acid structure which may be accurately designed and has self-assembly ability, thereby achieving multi-target interference, and may be used for inhibiting multiple gene expressions in a single pathway of disease occurrence or development, or simultaneously inhibiting multiple disease target gene expressions, possessing broad application prospects in multiple subject fields such as biology and chemistry.

### BRIEF DESCRIPTION OF THE DRAWINGS

FIG. 1 is a schematic diagram of a targeting nucleic acid unit X-type.

FIG. 2 is a schematic diagram of a flanking nucleic acid unit H-type.

FIG. 3 is a schematic diagram of a capping-end nucleic acid unit C-type.

FIG. 4 is a plane and ring schematic diagram of an R-structure polymeric nucleic acid. The upper diagram is a plane schematic diagram of the R-structure polymeric nucleic acid. The lower diagram is a ring schematic diagram of the R-structure polymeric nucleic acid (left: R=(Xi)4; and right: R=(Xi)6).

FIG. 5 is a plane schematic diagram of an L-structure polymeric nucleic acid.

FIG. 6 is a plane schematic diagram of a Cr-structure polymeric nucleic acid.

FIG. 7 is a single-gene relative expression level.

FIG. 8 is a multi-gene relative expression level.

FIG. 9 is a multi-gene relative expression level (n=4).

### DETAILED DESCRIPTION OF THE EMBODIMENTS

Unless otherwise specified, experiment methods used in the following embodiments are conventional methods.

Unless otherwise specified, materials, reagents and the like used in the following embodiments may be obtained from commercial sources.

### Embodiment 1. Design and synthesis of nucleic acid units and polymeric nucleic acids

I. Design of Nucleic Acid Unit

The present disclosure designs three types of nucleic acid units which are respectively named as an X-type targeting nucleic acid unit, an H-type flanking nucleic acid unit, and a C-type capping-end nucleic acid unit. These nucleic acid units may be freely combined into polymeric nucleic acids in various structures.

1. Targeting Nucleic Acid Unit: X-Type

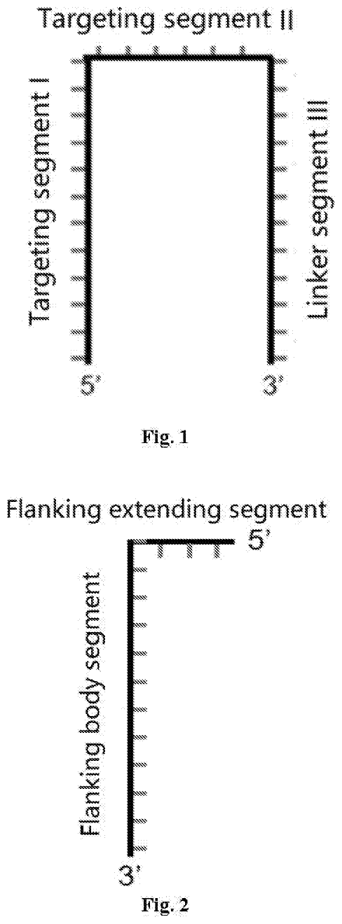

A structure of the X-type targeting nucleic acid unit is as follows: 5i-T1-T2-A3-3′. Herein T1 is a targeting segment I, T2 is a targeting segment II, A3 is a linker segment III, the T1 and T2 forms a sequence complementary to a target gene sequence. In certain specific cases, a region complementary to the target gene sequence may be extended to a partial sequence of the A3. A length of the X-type targeting nucleic acid unit is 15-50 nt, preferably 24-36 nt. Herein a length of the T1 is 5-24 nt, a length of the T2 is 1-20 nt, and a length of the A3 is 5-24 nt. The schematic diagram of the X-type targeting nucleic acid unit is as shown in FIG. 1.

2. Flanking Nucleic Acid Unit: H-Type

A structure of the H-type flanking nucleic acid unit is as follows: 3′-Hx-Hy-5′ or 3′-Hy-Hx-5′. Herein Hx is a flanking body segment, and Hy is a flanking extending segment (the Hy may not exist). The Hx and Hy are connected by a phosphate diester bond. The Hy is positioned at 5′-end or 3′-end of the Hx. The Hx is complementary-paired with the T1 segment or the A3 segment of the X unit; and the Hy is complementary-paired with one, two or more continuous nucleotides, from the 5′-end or the 3′-end, of the T2 segment of the X unit. A length of the Hy may be 2-6 nt or longer. The schematic diagram of the H-type Flanking nucleic acid unit is as shown in FIG. 2.

3. Capping-End Nucleic Acid Unit: C-Type

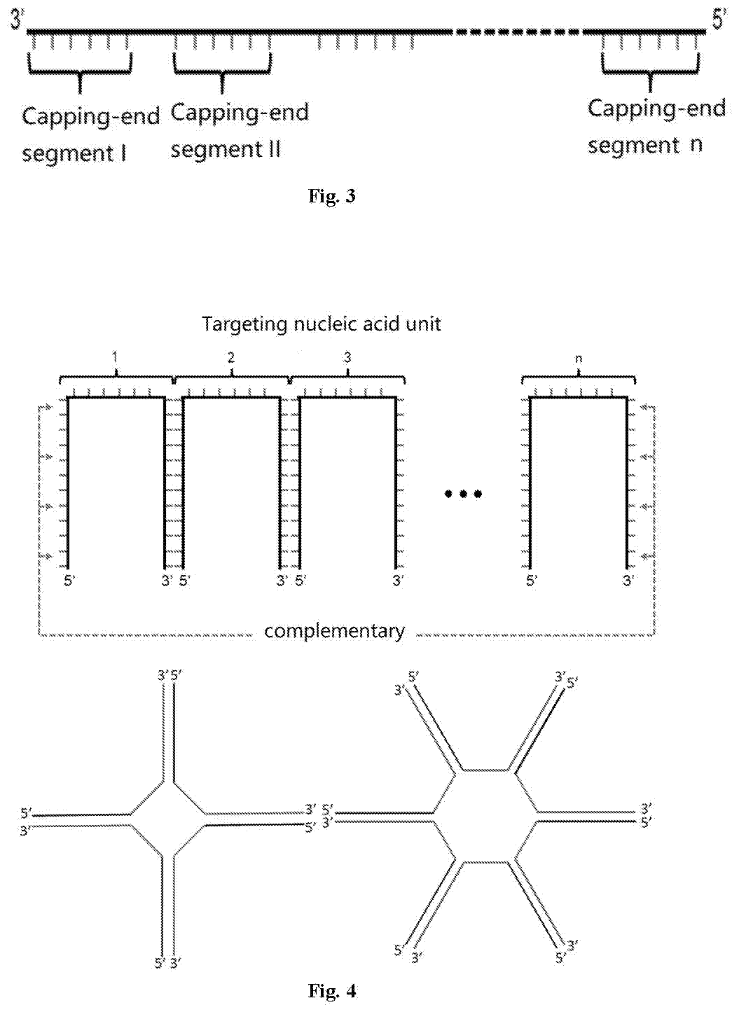

A structure of the C-type capping-end nucleic acid unit is as follows: 3′-C1-C2- . . . Cn-5′. Herein C1 is a capping-end segment 1, and reverse-complementary to the T2 segment of the X1 unit; C2 is a capping-end segment 2, and reverse-complementary to the T2 segment of the X2 unit, and so on, Cn is a capping-end segment n, and reverse-complementary to the T2 segment of the Xn unit. The schematic diagram of the C-type capping-end nucleic acid unit is as shown in FIG. 3.

II. Design of Polymeric Nucleic Acid Molecules

The present disclosure designs three structures of polymeric nucleic acids according to the three nucleic acid units in the step I, which are respectively named as a R-structure polymeric nucleic acid, an L-structure polymeric nucleic acid and a Cr-structure polymeric nucleic acid. These structures may be simultaneously targeted to the different sites of the same gene, and may also be simultaneously targeted to the different sites of the different genes, to interfere with the expression of the target gene.

1. Polymeric Nucleic Acid Structure I: R-Structure

A structure of the R-structure polymeric nucleic acid is as follows: (Xi)n. Herein Xi is a targeting nucleic acid unit. n is an integer greater than or equal to 3, preferably the integer of 3-8, more preferably 4, 5 and 6.

The n X-type targeting nucleic acid units are respectively named as an X1 unit, an X2 unit, an X3 unit, and so on, an Xn-1 unit, and an Xn unit.

In the R-structure polymeric nucleic acid of the present disclosure, each targeting nucleic acid unit forms a double-stranded region with the other two targeting nucleic acid units in the polymeric nucleic acid except for itself through sequence complementation, namely the adjacent X units are connected by complementary pairing of the T1 segment and the A3 segment, specifically, the A3 segment of the X1 unit is complementary-paired with the T1 segment of the X2 unit, the A3 segment of the X2 unit is complementary-paired with the T1 segment of the X3 unit, and so on, the A3 segment of the Xn unit is complementary-paired with the T1 segment of the X1 unit (the T1 segment of each targeting nucleic acid unit is complementary-paired with the A3 segment of the adjacent targeting nucleic acid unit thereof, and the T1 segment of each targeting nucleic acid unit is not complementary-paired with other targeting nucleic acid unit sequences), thereby a cyclic secondary structure is formed. In the cyclic structure, each targeting nucleic acid unit has the same structure and length.

Each targeting nucleic acid unit may be combined with a specific sequence of the target gene through the T1 and T2 thereof, thereby the expression of the target gene is controlled.

The plane and ring schematic diagram of the R-structure polymeric nucleic acid is as shown in FIG. 4.

2. Polymeric Nucleic Acid Structure II: L-Structure

A structure of the L-structure polymeric nucleic acid is as follows: H1-(Xi)n-Hn. Herein H1 and Hn are flanking nucleic acid units, Xi is the targeting nucleic acid unit. n is an integer greater than or equal to 3, preferably the integer of 3-8, more preferably 4, 5 and 6.

The n X-type targeting nucleic acid units are respectively named as an X1 unit, an X2 unit, an X3 unit, and so on, an Xn-1 unit, and an Xn unit.

In the L-structure polymeric nucleic acid of the present disclosure, the A3 segment of the X1 unit is complementary-paired with the T1 segment of the X2 unit, the A3 segment of the X2 unit is complementary-paired with the T1 segment of the X3 unit, and so on, the A3 segment of the Xn-1 unit is complementary-paired with the T1 segment of the Xn unit; and the H1 unit is complementary-paired with the T1 segment of the X1 unit. A complementary region of the H1 unit and the X1 unit may also be extended to a part or all of the T2 segment of the X1 unit; the Hn unit is complementary-paired with the A3 segment of the Xn unit, and a complementary region of the Hn unit and the Xn unit may also be extended to a part or all of the T2 segment of the Xn unit. The 5′-end of the H1 unit or the 3′-end of the Hn unit further includes a Hy segment, the Hy segment is complementary-paired with one, two or more continuous nucleic acid molecules, from the 5′-end or the 3′-end, of the targeting segment II of the X1 unit or the Xn unit.

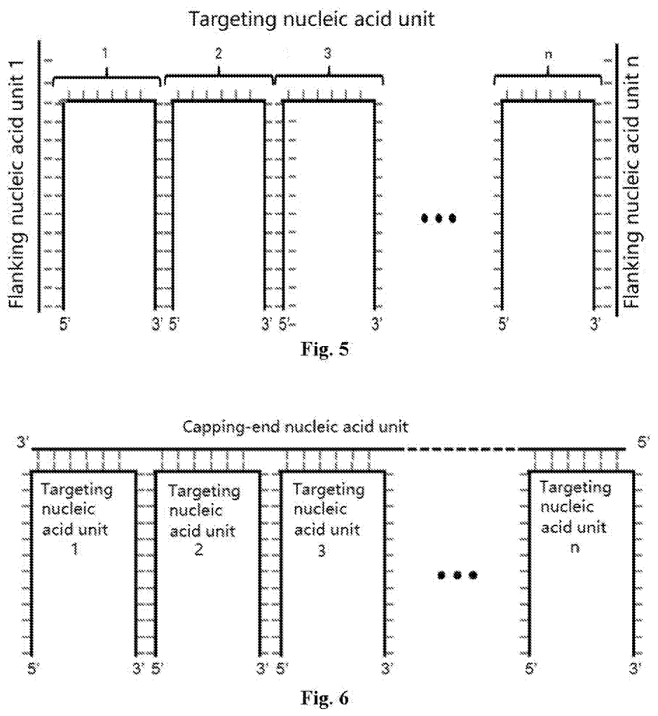

The plane schematic diagram of the L-structure polymeric nucleic acid is as shown in FIG. 5.

3. Polymeric Nucleic Acid Structure III: Cr-Structure

A structure of the Cr-structure polymeric nucleic acid is as follows: (Xi)n-C. Herein Xi is a targeting nucleic acid unit, and C is a capping-end nucleic acid unit. n is an integer greater than or equal to 3, preferably the integer of 3-8, more preferably 4, 5 and 6.

The n X-type targeting nucleic acid units are respectively named as an X1 unit, an X2 unit, an X3 unit, and so on, an Xn-1 unit, and an Xn unit.

In the Cr-structure polymeric nucleic acid of the present disclosure, the A3 segment of the X1 unit is complementary-paired with the T1 segment of the X2 unit, the A3 segment of the X2 unit is complementary-paired with the T1 segment of the X3 unit, and so on, the A3 segment of the Xn-1 unit is complementary-paired with the T1 segment of the Xn unit; the capping-end nucleic acid unit C is formed by a capping-end segment 1, a capping-end segment 2, and so on, a capping-end segment n, the capping-end segment 1 is complementary-paired with the T2 segment of the X1 unit, the capping-end segment 2 is complementary-paired with the T2 segment of the X2 unit, and so on, the capping-end segment n is complementary-paired with the T2 segment of the Xn unit, the capping-end segment 1, the capping-end segment 2, and so on, and the capping-end segment n are connected into a single-stranded nucleic acid through a phosphate diester bond.

The plane schematic diagram of the Cr-structure polymeric nucleic acid is as shown in FIG. 6.

III. Synthetic Method of Polymeric Nucleic Acid and Modification Method of Nucleic Acid Unit

1. Synthetic Method of Polymeric Nucleic Acid

The nucleic acid units of the present disclosure are annealed each other in a sequence specificity mode, the complementarity thereof promotes self-assembly of this polymeric nucleic acid, and a polymeric nucleic acid molecule with a secondary structure is formed. The specific synthetic method is as follows:

1) the nucleic acid units required by a polymeric nucleic acid structure is synthesized; and

2) the nucleic acid units are placed in an annealing condition, and annealed mutually, to form a secondary structure body by the self-assembly.

A reaction system for annealing is a system obtained by uniformly mixing each nucleic acid unit in equal molar weight, RNA annealing buffer (5X) (Biyuntian Annealing Buffer for RNA oligos (5X), R0051) and water (DEPC water).

A reaction condition for annealing is that a temperature is 90 DEG C in a polymerase chain reaction (PCR) meter, it is adequately denaturized in 2 min, and then the temperature in the PCR meter is reduced to 25 DEG C so that it is annealed.

2. Modification Method of the Nucleic Acid Units

In order to increase stability and biological activity of a nuclease, suitable modifications of nucleotide sugar, base and phosphate moieties may be introduced into the nucleic acid, including chemical modification of ribose, such as a 2′ ribose hydroxyl group is substituted by a halogen group or an O-alkyl group, the alkyl group includes a methyl, an ethyl, a propyl or a methylethyl and the like, such nucleic acid modification may not reduce the interference activity of the polymeric nucleic acid. The nucleic acid units designed by the present disclosure include the following modifications.

1) The ribose 2-site hydroxyl groups of 5-9 continuous nucleotides, from the first nucleotide at the 3′-end, of the linker segment A3 in the targeting nucleic acid unit are substituted by the halogen group or the O-alkyl group.

2) The ribose 2-site hydroxyl groups of 5-9 continuous nucleotides, from the first nucleotide at the 3′-end, of the flanking body segment of the flanking nucleic acid unit are substituted by the halogen group or the O-alkyl group; or the ribose 2-site hydroxyl groups of 8-30 continuous nucleotides, from the first nucleotide at the 3′-end, of the flanking nucleic acid unit are substituted by the halogen group or the O-alkyl group. Preferably, the ribose 2-site hydroxyl groups of 14-18 continuous nucleotides, from the first nucleotide at the 3′-end, of the flanking nucleic acid unit are substituted by the halogen group or the O-alkyl group.

3) The ribose 2-site hydroxyl groups of 2-6 continuous nucleotides, from the 5′-end to the 3′-end, of each capping-end segment of the capping-end nucleic acid unit are substituted by the halogen group or the O-alkyl group.

Embodiment 2. Preparation of polymeric nucleic acid and application thereof in interference of target gene expression

(I) R-structure polymeric nucleic acid: inhibition experiment targeting to different regions of a same gene

1. Polymeric Nucleic Acid

A polymeric nucleic acid in the following structure is prepared, a targeting nucleic acid unit of each structure is respectively targeting to 4, 5 or 6 different regions of VEGFA and TP53 genes.

A represents that T1 and A3 segments of the targeting nucleic acid unit are 12 nt, and a T2 segment is 6 nt.

B represents that T1 and A3 segments of the targeting nucleic acid unit are 12 nt, and a T2 segment is 5 nt.

C represents that T1 and A3 segments of the targeting nucleic acid unit are 12 nt, and a T2 segment is 3 nt.

D represents that T1 and A3 segments of the targeting nucleic acid unit are 11 nt, and a T2 segment is 6 nt.

E represents that T1 and A3 segments of the targeting nucleic acid unit are 10 nt, and a T2 segment is 6 nt.

While n is 4, the annealing system is as follows (a total volume is 100 μL): 20 μL of each nucleic acid unit solution (the concentration is 100 μM), 15 μL of the RNA annealing buffer (5X), and 5 μL of the DEPC water.

While n is 5, the annealing system is as follows (the total volume is 150 μL): 20 μL of each nucleic acid unit solution (the concentration is 150 μM), 30 μL of the RNA annealing buffer (5X), and 20 μL of the DEPC water.

While n is 6, the annealing system is as follows (the total volume is 200 μL): 20 μL of each nucleic acid unit solution (the concentration is 200 μM), 40 μL of the RNA annealing buffer (5X), and 40 μL of the DEPC water.

The polymeric nucleic acid of which the target gene is VEGFA is the following 1)-11):

1) VEGFA-R6-A (R-structure, n=6): an X unit sequence thereof is sequences 42-47 in Table 1 successively from X1-X6.

2) VEGFA-R6-B (R-structure, n=6): an X unit sequence thereof is sequences 48-53 in Table 1 successively from X1-X6.

3) VEGFA-R6-C(R-structure, n=6): an X unit sequence thereof is sequences 54-59 in Table 1 successively from X1-X6.

4) VEGFA-R5-A (R-structure, n=5): an X unit sequence thereof is sequences 42, 43, 45, 46 and 47 in Table 1 successively from X1-X5.

5) VEGFA-R5-B (R-structure, n=5): an X unit sequence thereof is sequences 48, 49, 51, 52 and 53 in Table 1 successively from X1-X5.

6) VEGFA-R5-C(R-structure, n=5): an X unit sequence thereof is sequences 54, 55, 57, 58 and 59 in Table 1 successively from X1-X5.

7) VEGFA-R4-A (R-structure, n=4): an X unit sequence thereof is sequences 42, 45, 46 and 47 in Table 1 successively from X1-X4.

8) VEGFA-R4-B (R-structure, n=4): an X unit sequence thereof is sequences 48, 51, 52 and 53 in Table 1 successively from X1-X4.

9) VEGFA-R4-C(R-structure, n=5): an X unit sequence thereof is sequences 54, 57, 58 and 59 in Table 1 successively from X1-X4.

10) VEGFA-R6-D (R-structure, n=6): an X unit sequence thereof is sequences 60-65 in Table 1 successively from X1-X6.

11) VEGFA-R6-E (R-structure, n=6): an X unit sequence thereof is sequences 66-71 in Table 1 successively from X1-X6.

The polymeric nucleic acid of which the target gene is TP53 is the following 1)-11):

1) TP53-R6-A (R-structure, n=4): an X unit sequence thereof is sequences 72-77 in Table 2 successively from X1-X6.

2) TP53-R6-B (R-structure, n=6): an X unit sequence thereof is sequences 78-83 in Table 2 successively from X1-X6.

3) TP53-R6-C(R-structure, n=6): an X unit sequence thereof is sequences 84-89 in Table 2 successively from X1-X6.

4) TP53-R5-A (R-structure, n=5): an X unit sequence thereof is sequences 73-77 in Table 2 successively from X1-X5.

5) TP53-R5-B (R-structure, n=5): an X unit sequence thereof is sequences 79-83 in Table 2 successively from X1-X5.

6) TP53-R5-C(R-structure, n=5): an X unit sequence thereof is sequences 85-89 in Table 2 successively from X1-X5.

7) TP53-R4-A (R-structure, n=4): an X unit sequence thereof is sequences 74-77 in Table 2 successively from X1-X4.

8) TP53-R4-B (R-structure, n=4): an X unit sequence thereof is sequences 80-83 in Table 2 successively from X1-X4.

9) TP53-R4-C(R-structure, n=5): an X unit sequence thereof is sequences 86-89 in Table 2 successively from X1-X4.

10) TP53-R6-D (R-structure, n=6): an X unit sequence thereof is sequences 90-95 in Table 2 successively from X1-X6.

11) TP53-R6-E (R-structure, n=6): an X unit sequence thereof is sequences 96-101 in Table 2 successively from X1-X6.

TABLE 1
Sequences of nucleic acid units

| Target | | |
|---|---|---|
| gene | Sequence (5′-3′) | SEQ ID NO: |
| VEGFA | AGCAGAAAGUUCAUGGUUUCUUGggugcau | 42 |
| VEGFA | AUGCACCCAAGACAGCAGAGAUCgaguaca | 43 |
| VEGFA | UGUACUCGAUCUCAUCAGGGUGGacaucuu | 44 |
| VEGFA | AAGAUGUCCACCAGGGUCAUGCGgaucaaa | 45 |
| VEGFA | UUUGAUCCGCAUAAUCUGGGCCAgcacaua | 46 |
| VEGFA | UAUGUGCUGGCCUUGGUGGAACUuucugcu | 47 |
| VEGFA | AGCAGAAAGUUCAUGGUUCUUGggugcau | 48 |
| VEGFA | AUGCACCCAAGACAGCAAGAUCgaguaca | 49 |
| VEGFA | UGUACUCGAUCUCAUCAGGUGGacaucuu | 50 |
| VEGFA | AAGAUGUCCACCAGGGUAUGCGgaucaaa | 51 |
| VEGFA | UUUGAUCCGCAUAAUCUGGCCAgcacaua | 52 |
| VEGFA | UAUGUGCUGGCCUUGGUGAACUuucugcu | 53 |
| VEGFA | AGCAGAAAGUUCAUGUCUUGggugcau | 54 |
| VEGFA | AUGCACCCAAGACAGAGAUCgaguaca | 55 |
| VEGFA | UGUACUCGAUCUCAUGGUGGacaucuu | 56 |
| VEGFA | AAGAUGUCCACCAGGAUGCGgaucaaa | 57 |
| VEGFA | UUUGAUCCGCAUAAUGGCCAgcacaua | 58 |
| VEGFA | UAUGUGCUGGCCUUGGAACUuucugcu | 59 |
| VEGFA | AGCAGAAAGUUCAUGGUCUUGggugcau | 60 |
| VEGFA | AUGCACCCAAGACAGCAGAUCgaguaca | 61 |
| VEGFA | UGUACUCGAUCUCAUCAGUGGacaucuu | 62 |
| VEGFA | AAGAUGUCCACCAGGGUUGCGgaucaaa | 63 |
| VEGFA | UUUGAUCCGCAUAAUCUGCCAgcacaua | 64 |
| VEGFA | UAUGUGCUGGCCUUGGUAACUuucugcu | 65 |
| VEGFA | AGCAGAAAGUUCAUGGUUGggugcau | 66 |
| VEGFA | AUGCACCCAAGACAGCAUCgaguaca | 67 |
| VEGFA | UGUACUCGAUCUCAUCUGGacaucuu | 68 |
| VEGFA | AAGAUGUCCACCAGGGGCGgaucaaa | 69 |
| VEGFA | UUUGAUCCGCAUAAUCCCAgcacaua | 70 |
| VEGFA | UAUGUGCUGGCCUUGGACUuucugcu | 71 |

TABLE 2
Nucleic acid unit sequences

| Target | | |
|---|---|---|
| gene | Sequence (5′-3′) | SEQ ID NO: |
| TP53 | UGUGGAAUCAACCCACAGUUUGCgugugga | 72 |
| TP53 | UCCACACGCAAAUUUCCUACAGAaacacuu | 73 |
| TP53 | AAGUGUUUCUGUCAUCCAACUACaugugua | 74 |
| TP53 | UACACAUGUAGUUGUAGUUGGUAaucuacu | 75 |
| TP53 | AGUAGAUUACCACUGGAGUCUCCgcaagaa | 76 |
| TP53 | UUCUUGCGGAGAUUCUCUGUUGAuuccaca | 77 |
| TP53 | UGUGGAAUCAACCCACAUUUGCgugugga | 78 |
| TP53 | UCCACACGCAAAUUUCCACAGAaacacuu | 79 |
| TP53 | AAGUGUUUCUGUCAUCCACUACaugugua | 80 |
| TP53 | UACACAUGUAGUUGUAGUGGUAaucuacu | 81 |
| TP53 | AGUAGAUUACCACUGGAUCUCCgcaagaa | 82 |
| TP53 | UUCUUGCGGAGAUUCUCGUUGAuuccaca | 83 |
| TP53 | UGUGGAAUCAACCCAUUUGCgugugga | 84 |
| TP53 | UCCACACGCAAAUUUACAGAaacacuu | 85 |
| TP53 | AAGUGUUUCUGUCAUACUACaugugua | 86 |
| TP53 | UACACAUGUAGUUGUUGGUAaucuacu | 87 |
| TP53 | AGUAGAUUACCACUGUCUCCgcaagaa | 88 |
| TP53 | UUCUUGCGGAGAUUCGUUGAuuccaca | 89 |
| TP53 | UGUGGAAUCAACCCACAUUGCgugugga | 90 |
| TP53 | UCCACACGCAAAUUUCCCAGAaacacuu | 91 |
| TP53 | AAGUGUUUCUGUCAUCCCUACaugugua | 92 |
| TP53 | UACACAUGUAGUUGUAGGGUAaucuacu | 93 |
| TP53 | AGUAGAUUACCACUGGACUCCgcaagaa | 94 |
| TP53 | UUCUUGCGGAGAUUCUCUUGAuuccaca | 95 |
| TP53 | UGUGGAAUCAACCCACUGCgugugga | 96 |
| TP53 | UCCACACGCAAAUUUCAGAaacacuu | 97 |
| TP53 | AAGUGUUUCUGUCAUCUACaugugua | 98 |
| TP53 | UACACAUGUAGUUGUAGUAaucuacu | 99 |
| TP53 | AGUAGAUUACCACUGGUCCgcaagaa | 100 |
| TP53 | UCUUGCGGAGAUUCUUGAuuccaca | 101 |

Note:
lower case letters represent that the nucleotide ribose is modified by 2′-O-methyl ribose.
The underlined sequence is a target sequence.

2. Inhibition Experiment

5×10⁵ HeLa cells (an ATCC number is CRL-1958) are inoculated in a 12-pore culture plate of a DMEM culture medium containing 10% of fetal bovine serum, and the polymeric nucleic acid prepared in the step 1 is used to respectively transfect the HeLa cells: after the polymeric nucleic acid is mixed with a transfection reagent and buffer (GUANGZHOU RIBOBIO CO., LTD., named as riboFECT™ CP Buffer, and Article No. C10511-1), it is added to the cell culture medium, a volume of each pore is 1 mL, so that the final transfection concentration of the polymeric nucleic acid is 50 nM, and the culture plate is placed in an incubator with 5% of CO₂ and 37 DEG C of a constant temperature, and cultured for 48 h. The transfection reagent and specific steps of transfection refer to a method in riboFect™ (GUANGZHOU RIBOBIO CO., LTD).

In addition to a test group, a negative control group (NC) and an untreated control group (NT) are also set for each time of cell plating. There are 3 replicates in the test group and the control groups. A control sequence of the NC group is siRNA, and is a double-stranded RNA molecule obtained by complementary-binding of the following two single-stranded RNA molecules: 5′-UUCUCCGAACGUGUCACGUdTdT-3′(SEQ ID NO:102) and 5′-ACGUGACACGUUCGGAGAAdTdT-3. (SEQ ID NO:103).

In 37 DEG C and 5% of the CO₂, after being incubated for 48 h, transfected cells are collected, RNAs of the transfected cells are extracted by a Trizol method and a real-time quantitative PCR is performed, an mRNA expression level of the target gene is detected, q-PCR is repeated 3 times, and all of results are represented by an average value±SD. Sequences of real-time quantitative PCR primers for detecting the target genes are as shown in the attached table.

Detection results are as shown in Table 3. It is indicated from an experiment result that the above polymer molecules in different structures may efficiently inhibit the expression of the target gene.

TABLE 3
mRNA relative expression level

| | VEGFA | TP53 |
|---|---|---|
| R6-A | 0.05 | 0.10 |
| R6-B | 0.02 | 0.06 |
| R6-C | 0.13 | 0.21 |
| R5-A | 0.06 | 0.06 |
| R5-B | 0.03 | 0.02 |
| R5-C | 0.12 | 0.12 |
| R4-A | 0.04 | 0.04 |
| R4-B | 0.06 | 0.01 |
| R4-C | 0.23 | 0.13 |
| R6-D | 0.06 | 0.22 |
| R6-E | 0.06 | 0.07 |

Note:
the negative control expression level is 1.
(II) R-structure polymeric nucleic acid: low-concentration inhibition experiment

1. Polymeric Nucleic Acid

A step of preparing an X unit sequence of the polymeric nucleic acid VEGFA-R6-A and an X unit sequence of the polymeric nucleic acid TP53-R6-A is the same as the step (I); and an X unit sequence of the polymeric nucleic acid P65-R6-A prepared is successively sequences 15, 16, 17, 23, 24 and 25 in Table 5 from X1-X6.

2. Inhibition experiment

Only the final transfection concentration of the polymeric nucleic acid in the inhibition experiment in the step (I) is changed to 1 nM, and the other steps are not changed.

A detection result is as shown in Table 4. It is indicated from an experiment result that the polymeric nucleic acid of the present disclosure with the low-concentration may also achieve a purpose of reducing the expression level of the target gene.

TABLE 4
mRNA relative expression level

| | VEGFA | TP53 | P65 |
|---|---|---|---|
| R6-A (1 nM) | 0.53 | 0.64 | 0.51 |

Note:
the negative control expression level is 1.
(III) Three structures of polymeric nucleic acids: inhibition experiment targeted to different regions of a same gene

1. Polymeric Nucleic Acids

The polymeric nucleic acids in the following structures are prepared, a targeting nucleic acid unit of each structure is respectively targeted to 4 or 6 different regions of PPIB or P65 gene.

The polymeric nucleic acid of which the target gene is PPIB is the following 1)-5):

1) PPIB-R4 (R-structure, n=4): an X unit sequence thereof is successively sequences 1-4 in Table 5 from X1-X4.

2) PPIB-L4 (L-structure, n=4): an X unit sequence thereof is successively sequences 1-3 and the sequence 5 in Table 5 from X1-X4, an H1 unit sequence thereof is the sequence 20, and an H4 unit sequence thereof is the sequence 21.

3) PPIB-Cr4 (Cr-structure, n=4): an X unit sequence thereof is successively sequences 1-4 in Table 5 from X1-X4, and a C unit sequence thereof is the sequence 8.

4) PPIB-R6 (R-structure, n=6): an X unit sequence thereof is successively sequences 2, 3, 9, 10, 11 and 12 in Table 5 from X1-X6.

5) PPIB-L6 (L-structure, n=6): an X unit sequence thereof is successively sequences 2, 3, 9, 10, 11 and 13 in Table 5 from X1-X6, an H1 unit sequence thereof is the sequence 6, and an H6 unit sequence thereof is the sequence 14.

The polymeric nucleic acid of which the target gene is P65 is the following 1)-5):

1) P65-R4 (R-structure, n=4): an X unit sequence thereof is successively sequences 15-18 in Table 5 from X1-X4.

2) P65-L4 (L-structure, n=4): an X unit sequence thereof is successively sequences 15-17 and the sequence 19 in Table 5 from X1-X4, an H1 unit sequence thereof is the sequence 6, and an H4 unit sequence thereof is the sequence 7.

3) P65-Cr4 (Cr-structure, n=4): an X unit sequence thereof is successively sequences 15-18 in Table 5 from X1-X4, and a C unit sequence thereof is the sequence 22.

4) P65-R6 (R-structure, n=6): an X unit sequence thereof is successively sequences 15, 16, 17, 23, 24 and 25 in Table 5 from X1-X6.

5) P65-L6 (L-structure, n=6): an X unit sequence thereof is successively sequences 15, 16, 17, 23, 24 and 26 in Table 5 from X1-X6, an H1 unit sequence thereof is the sequence 20, and an H6 unit sequence thereof is the sequence 27.

TABLE 5
Sequences of nucleic acid units

| Target | | |
|---|---|---|
| gene | Sequence (5′-3′) | SEQ ID NO: |
| PPIB | AGAUGCUCUUUCCUCCUGCAAGGuguauuu | 1 |
| PPIB | AAAUACACCUUGACGGUGGAUGAagaugua | 2 |
| PPIB | UACAUCUUCAUCUCCAAUUCUCUucggaaa | 3 |
| PPIB | UUUCCGAAGAGACCAAAGGAAAGagcaucu | 4 |
| PPIB | UUUCCGAAGAGACCAAAGUCUACgagaaag | 5 |
| PPIB | GGAGGAAAGagcaucu | 6 |
| PPIB | cuuucucguagacuuu | 7 |
| PPIB | cuuuGGauugGAcaccGUcaggAG | 8 |
| PPIB | AGAUGCUCUUUCCUCCUGCAUGAaggugcu | 9 |
| PPIB | AGCACCUUCAUGUUGCGUCAAGGuguauuu | 10 |
| PPIB | UUUCCGAAGAGACCAAAGCGUGUaaucaag | 11 |
| PPIB | CUUGAUUACACGAUGGAAGAAAGagcaucu | 12 |
| PPIB | CUUGAUUACACGAUGGAAUCUACgagaaag | 13 |
| PPIB | cuuucucguagauucc | 14 |
| P65 | UGUGUAGCCAUUGAUCUUGCAUCaugaaga | 15 |
| P65 | UCUUCAUGAUGCUCUUGAAUACCaccaaga | 16 |
| P65 | UCUUGGUGGUAUCUGUGCUCGUCaccggau | 17 |
| P65 | AUCCGGUGACGAUCGUCUAAUGGcuacaca | 18 |
| P65 | AUCCGGUGACGAUCGUCUACACAucgguaa | 19 |
| P65 | GAUCAAUGGcuacaca | 20 |
| P65 | uuaccgauguguagac | 21 |
| P65 | agacGAgcacAGucaaGAaagaUC | 22 |
| P65 | AUCCGGUGACGAUCGUCUUCAGGagaugaa | 23 |
| P65 | UUCAUCUCCUGAAAGGAGAUCAGcuccuaa | 24 |
| P65 | UUAGGAGCUGAUCUGACUAAUGGcuacaca | 25 |
| P65 | UUAGGAGCUGAUCUGACUACACAucgguaa | 26 |
| P65 | uuaccgauguguaguc | 27 |

Note:
lower case letters represent that the nucleotide ribose is modified by 2-O-methyl ribose.
The underlined sequence is a target sequence.
X unit: the 1-12th sites are T1; the 13-18th sites are T2; and the 19-30th sites are A3.
H1 unit: the 1-4th sites are Hy; and the 5-16th sites are Hx.
H4 unit: the 1-12th sites are Hx; and the 13-16th sites are Hy.
C unit: the 1-6th sites are C4, the 7-12th sites are C3, the 13-18th sites are C2, and the 19-24th sites are C1.

2. Inhibition Experiment

Steps of the inhibition experiment are the same as the step (I).

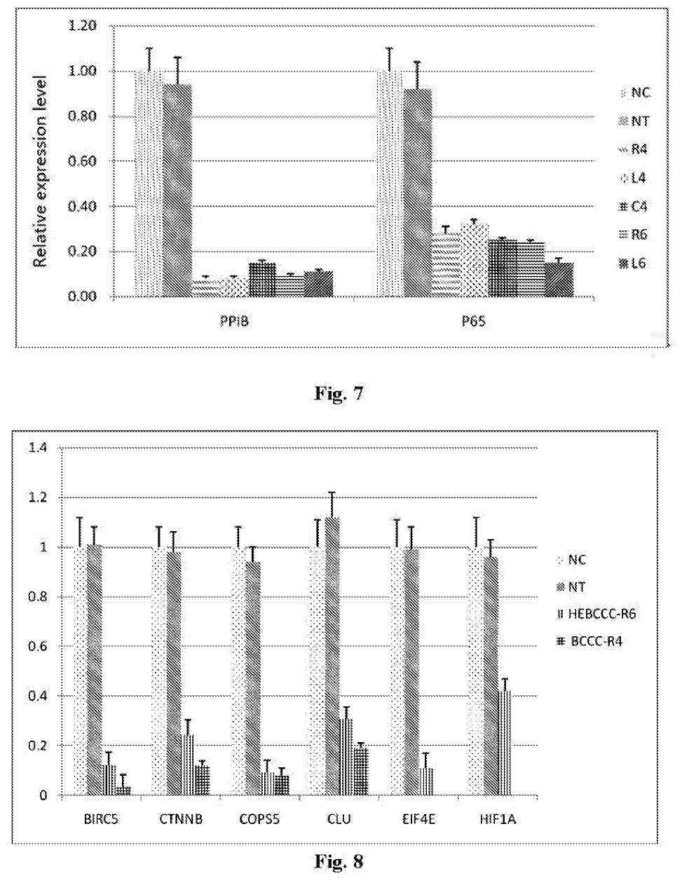

An inhibition result is as shown in FIG. 7. It is indicated from the result that 5 structures of the polymeric nucleic acids effectively inhibit the expression of the target genes. Inhibition levels in allusion to the target gene PP/B are above 80%, and the inhibition levels in allusion to the target gene P65 are above 75%.

(IV) Inhibition experiment of R-structure polymeric nucleic acid targeted to different genes (n=4 and 6)

1. Polymeric Nucleic Acid

A polymeric nucleic acid in the following R-structure is prepared, and the polymeric nucleic acid is targeted to 4 or 6 different target genes.

BCCC-R4: the target genes are BIRC5, CTNNB, COPS5 and CLU, an X unit sequence thereof is successively sequences 28-31 in Table 3 from X1-X4.

BCCCEH-R6: the target genes are BIRC5, CTNNB, COPS5, CLU, EIF4E and HIF1A, an X unit sequence thereof is successively sequences 32, 33, 28, 29, 30 and 34 in Table 6 from X1-X6.

2. Inhibition Experiment

Steps of the inhibition experiment are the same as the step (I).

TABLE 6
Sequences of nucleic acid units

| Target | | |
|---|---|---|
| gene | Sequence (5′-3′) | SEQ ID NO: |
| BIRC5 | AGAAGAAACACUGGGCCAACUGCugaucuu | 28 |
| CTNNB1 | AAGAUCAGCAGUCUCAUUUGGUCaggucuu | 29 |
| COPS5 | AAGACCUGACCAGUGGUAUUUGAcucugau | 30 |
| CLU | AUCAGAGUCAAAGAGCUUAGUGUuucuucu | 31 |
| HIF1A | UCAAGUUGCUGGUCAUCAGGAGGuugcuaa | 32 |
| EIF4E | UUAGCAACCUCCUGAUUAAGUGUuucuucu | 33 |
| CLU | AUCAGAGUCAAAGAGCUUCCAGCaacuuga | 34 |

Note:
lower case letters represent that the nucleotide ribose is modified by 2-O-methyl ribose.
The underlined sequence is a target sequence.
X unit: the 1-12th sites are T1; the 13-18th sites are T2; and the 19-30th sites are A3.

A result of the inhibition experiment is as shown in FIG. 8. It is indicated from the result that 2 structures of the polymeric nucleic acids effectively inhibit the expressions of 4 or 6 target genes. Expression levels of the polymeric nucleic acid in R4-structure targeted to 4 genes are reduced below 0.2.

(V) Inhibition experiment of polymeric nucleic acids in R-structure, L-structure and Cr-structure targeted to different genes (n=4)

1. Polymeric Nucleic Acid

A polymeric nucleic acid in each following structure is prepared, and the polymeric nucleic acid is targeted to 4 different target genes.

SPPV-R4: the target genes are SOD1, PPIB, P65, and VEGFA, an X unit sequence thereof is successively sequences 35-38 in Table 7 from X1-X4.

SPPV-L4: the target genes are SOD1, PP/B, P65, and VEGFA, an X unit sequence thereof is successively sequences 35-38 in Table 7 from X1-X4, an H1 unit sequence thereof is the sequence 39, and an H4 unit sequence thereof is the sequence 40.

SPPV-Cr4: the target genes are SOD1, PPIB, P65, and VEGFA, an X unit sequence thereof is successively sequences 35-38 in Table 7 from X1-X4, and a C unit sequence thereof is the sequence 41.

2. Inhibition Experiment

It is the same as the inhibition experiment in the step (I).

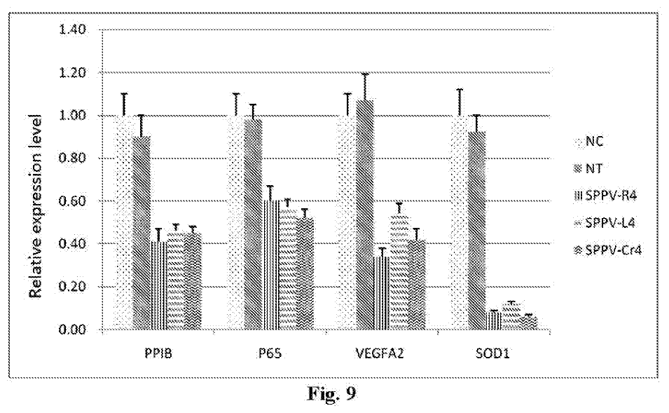

A result of the inhibition experiment is as shown in FIG. 9. It is indicated from the result that 3 structures of the polymeric nucleic acids effectively inhibit the expressions of 4 target genes. It is also discovered that in three polymeric structures, the inhibition effect of the polymeric nucleic acid in Cr4-structure is better, and an expression level of each target gene thereof is the lowest.

TABLE 7
Sequences of nucleic acid units

| Target | | |
|---|---|---|
| gene | Sequence (5′-3′) | SEQ ID NO: |
| SOD1 | UACUUUCUUCAUUUCCACCUGUUccaaaaa | 35 |
| PPIB | UUUUUGGAACAGUCUUUCUGAGAccuucaa | 36 |
| P65 | UUGAAGGUCUCAUAUGUCCAUGCagauuau | 37 |
| VEGFA | AUAAUCUGCAUGGUGAUGAUGAAgaaagua | 38 |
| H | GGAAAUGAAgaaagua | 39 |
| H4 | uacuuucuucaucauc | 40 |
| C | caucACgacaUAgaaaGAguggAA | 41 |

Note:
lower case letters represent that the nucleotide ribose is modified by 2-O-methyl ribose.
The underlined sequence is a target sequence.
X unit: the 1-12th sites are T1; the 13-18th sites are T2; and the 19-30th sites are A3.
H1 unit: the 1-4th sites are Hy; and the 5-16th sites are Hx.
H4 unit: the 1-12th sites are Hx; and the 13-16th sites are Hy.
C unit: the 1-6th sites are C4, the 7-12th sites are C3, the 13-18th sites are C2, and the 19-24th sites are C1.

Sequences of real-time quantitative PCR primers for detecting the target genes in each of the above experiments are as shown in Table 8.

TABLE 8
Sequences of real-time quantitative PCR
primers for detecting target genes

| | | SEQ |
|---|---|---|
| | | ID |
| Primer name | Primer sequence (5′-3′) | NO: |
| H-TP53-qPCR-F | TTGTGCCTGTCCTGGGAGAG | 104 |
| H-TP53-qPCR-R | GGAGAGGAGCTGGTGTTGTTG | 105 |
| H-HIF1A-qPCR-F | GCCCTAACGTGTTATCTGTC | 106 |
| H-HIF1A-qPCR-R | CGCTTTCTCTGAGCATTCTG | 107 |
| h-EIF4E-qPCR-F | GGAGGTTGCTAACCCAGAACAC | 108 |
| h-EIF4E-qPCR-R | GGAGATCAGCCGCAGGTTTG | 109 |
| h-VEGFA-qPCR-F | GAGGGCAGAATCATCACGAAG | 110 |
| h-VEGFA-qPCR-R | ACTCGATCTCATCAGGGTACTC | 111 |
| h-PPIB-qPCR-F | GGCAAGCATGTGGTGTTTGG | 112 |
| h-PPIB-qPCR-R | GGTTTATCCCGGCTGTCTGTC | 113 |
| h-p65-qPCR-F | GGGAAGGAACGCTGTCAGAG | 114 |
| h-p65-qPCR-R | TAGCCTCAGGGTACTCCATCA | 115 |
| h-SOD1-qPCR-F | GCAGGGCATCATCAATTTCG | 116 |
| h-SOD1-qPCR-R | GAATCCATGCAGGCCTTCAG | 117 |
| H-BIRC5-qPCR-F | AGAACTGGCCCTTCTTGGAG | 118 |
| H-BIRC5-qPCR-R | GAAACACTGGGCCAAGTCTG | 119 |
| H-CTNNB1-qPCR-F | GCTCGGGATGTTCACAACC | 120 |
| H-CTNNB1-qPCR-R | CCCTGCAGCTACTCTTTGG | 121 |
| H-COPS5-qPCR-F | TGGAATAAATACTGGGTGAATACG | 122 |
| H-COPS5-qPCR-R | GGCTTCTGACTGCTCTAAC | 123 |
| H-CLU-qPCR-F | CAAGGCGAAGACCAGTACTATC | 124 |
| H-CLU-qPCR-R | CAGTGACACCGGAAGGAAC | 125 |

### INDUSTRIAL APPLICATION

The present disclosure provides a new-type nucleic acid unit for constructing a polymeric nucleic acid and the polymeric nucleic acid for interfering with an expression of a target gene. The present disclosure is capable of, through designing and constructing the new-type nucleic acid unit and the self-assembled polymeric nucleic acid thereof, achieving multi-target interference, and may be used for inhibiting multiple gene expressions in a signaling pathway of disease occurrence or development, or simultaneously inhibiting multiple disease target gene expressions, possessing broad application prospects in multiple subject fields such as biology and chemistry. The polymeric nucleic acid may target multiple sequences simultaneously, herein the sequences may be located in one gene, or located in multiple genes. The advantages of the present disclosure include: 1) a high RNAi performance; 2) good stability; 3) reduction of an off-target rate; 4) enhanced ability to be introduced into cells; and 5) a modular design.

What is claimed is:

1. A polymeric nucleic acid molecule for interfering with an expression of a target gene, wherein the polymeric nucleic acid molecule is formed by n X-type nucleic acid molecules;
each of the X-type nucleic acid molecules is formed by a targeting segment I, a targeting segment II and a linker segment III successively from a 5′-end;
the targeting segment I of each of the X-type nucleic acid molecules is complementary-paired with the linker segment III of an adjacent X-type nucleic acid molecule thereof;
the targeting segment I and the targeting segment II of each of the X-type nucleic acid molecules is complementary-paired with a target gene;
a length of each of the X-type nucleic acid molecules is the same; and
the n is an integer greater than or equal to 3.

2. The polymeric nucleic acid molecule as claimed in claim 1, wherein the polymeric nucleic acid molecule further comprises a H-type nucleic acid molecule; the H-type nucleic acid molecule is formed by a H1-type nucleic acid molecule and a Hn-type nucleic acid molecule;
the n X-type nucleic acid molecules are respectively named as an X1 unit, an X2 unit, an X3 unit, and so on, an Xn-1 unit, and an Xn unit;
the linker segment III of the X1 unit is complementary-paired with the targeting segment I of the X2 unit, the linker segment III of the X2 unit is complementary-paired with the targeting segment I of the X3 unit, and so on, the linker segment III of the Xn-1 unit is complementary-paired with the targeting segment I of the Xn unit;
the H1-type nucleic acid molecule is complementary-paired with the targeting segment I of the X1 unit, and the Hn-type nucleic acid molecule is complementary-paired with the linker segment III of the Xn unit.

3. The polymeric nucleic acid molecule as claimed in claim 2, wherein
5′-end or 3′-end of the H-type nucleic acid molecule further comprises a Hy segment; and the H-type nucleic acid molecule is formed by an Hx segment and the Hy segment;
the Hx segment of the H1-type nucleic acid molecule is complementary-paired with the targeting segment I of the X1 unit; the Hx segment of the Hn-type nucleic acid molecule is complementary-paired with the linker segment III of the Xn unit; and
the Hy segment is complementary-paired with one, two or more continuous nucleic acid molecules of the targeting segment II of the X1 unit or the Xn unit from 5′-end or 3′-end.)

4. The polymeric nucleic acid molecule as claimed in claim 1, wherein the polymeric nucleic acid molecule further comprises a C-type nucleic acid molecule;
the C-type nucleic acid molecule is formed by n segments connected successively, the n segments are respectively reverse complementary sequences of the targeting segment II in the n X-type nucleic acid molecules.

5. The polymeric nucleic acid molecule as claimed in claim 1, the polymeric nucleic acid molecule further comprises a C-type nucleic acid molecule;
the C-type nucleic acid molecule is formed by n segments connected successively, the n segments are respectively reverse complementary sequences of the targeting segment II in the n X-type nucleic acid molecules;
wherein the X-type nucleic acid molecule, the H-type nucleic acid molecule and the C-type nucleic acid molecule are a single-stranded RNA molecule.

6. The polymeric nucleic acid molecule as claimed in claim 1, wherein
a length of the X-type nucleic acid molecule is 15-50 nt; preferably is 24-36 nt;
a length of the targeting segment I is 5-24 nt;
a length of the targeting segment II is 1-20 nt;
a length of the linker segment III is 5-24 nt; and
a sum of the lengths of the targeting segment I and the targeting segment II is 14-16 nt at least.

7. (canceled)

8. The polymeric nucleic acid molecule as claimed in claim 1, wherein the polymeric nucleic acid molecule at least comprises a modified nucleotide;
preferably, a modification of the modified nucleotide is a phosphoric acid backbone modification, a base modification and/or a ribose modification;
more preferably, the ribose modification is that a ribose 2-site hydroxyl group is substituted by a halogen group or an O-alkyl group;
further preferably, an alkyl of the O-alkyl group is a methyl, an ethyl, a propyl or a methylethyl.

9. (canceled)

10. (canceled)

11. (canceled)

12. The polymeric nucleic acid molecule as claimed in claim 8, wherein the ribose 2-site hydroxyl groups of 5-9 continuous nucleotides, from the first nucleotide at the 3′-end, of the linker segment III of the X-type nucleic acid molecule are substituted by the halogen group or the O-alkyl group;
or, the ribose 2-site hydroxyl groups of 5-9 continuous nucleotides, from the first nucleotide at the 3′-end, of the Hx segment of the H-type nucleic acid molecule are substituted by the halogen group or the O-alkyl group;
or, the ribose 2-site hydroxyl groups of 8-30 continuous nucleotides, from the first nucleotide at the 3′-end, of the H-type nucleic acid molecule are substituted by the halogen group or the O-alkyl group;
or, the ribose 2-site hydroxyl groups of 2-6 continuous nucleotides, from the first nucleotide at the 5′-end, of each segment in the C-type nucleic acid molecule complementary-paired with the targeting segment II of the X-type nucleic acid molecule are substituted by the halogen group or the O-alkyl group.)

13. The polymeric nucleic acid molecule as claimed in claim 12, wherein the ribose 2-site hydroxyl groups of 14-18 continuous nucleotides, from the first nucleotide at the 3′-end, of the H-type nucleic acid molecule are substituted by the halogen group or the O-alkyl group.)

14. The polymeric nucleic acid molecule as claimed in claim 1, wherein the n is 3 or 4 or 5 or 6 or 7 or 8.

15. (canceled)

16. The polymeric nucleic acid molecule as claimed in claim 1, wherein the number of the target gene is one or two or more, and the number of the target gene does not exceed n;
or, the number of the target gene is 1 or 4 or 6.)

17. The polymeric nucleic acid molecule as claimed in claim 1, wherein the target gene is at least one of the following genes: PPIB, p65, BIRC5, CTNNB, COPS5, CLU, EIF4E, HIF1A, TP53, VEGFA and SOD1.

18. The polymeric nucleic acid molecule as claimed in claim 17, wherein
the polymeric nucleic acid molecule for interfering with an expression of the target gene PP1B is the following a1)-a5):
a1) is formed by single-stranded RNA molecules shown in SEQ ID NO: 1, SEQ ID NO: 2, SEQ ID NO: 3 and SEQ ID NO: 4;
a2) is formed by single-stranded RNA molecules shown in SEQ ID NO: 1, SEQ ID NO: 2, SEQ ID NO: 3, SEQ ID NO: 4, SEQ ID NO: 20 and SEQ ID NO: 21;
a3) is formed by single-stranded RNA molecules shown in SEQ ID NO: 1, SEQ ID NO: 2, SEQ ID NO: 3, SEQ ID NO: 4 and SEQ ID NO: 8;
a4) is formed by single-stranded RNA molecules shown in SEQ ID NO: 2, SEQ ID NO: 3, SEQ ID NO: 9, SEQ ID NO: 10, SEQ ID NO: 11 and SEQ ID NO: 12;
a5) is formed by single-stranded RNA molecules shown in SEQ ID NO: 2, SEQ ID NO: 3, SEQ ID NO: 6, SEQ ID NO: 9, SEQ ID NO: 10, SEQ ID NO: 11, SEQ ID NO: 13 and SEQ ID NO: 14;
or, the polymeric nucleic acid molecule for interfering with an expression of the target gene P65 is the following b1)-b5):
b1) is formed by single-stranded RNA molecules shown in SEQ ID NO: 15, SEQ ID NO: 16, SEQ ID NO: 17 and SEQ ID NO: 18;
b2) is formed by single-stranded RNA molecules shown in SEQ ID NO: 6, SEQ ID NO: 7, SEQ ID NO: 15, SEQ ID NO: 16, SEQ ID NO: 17 and SEQ ID NO: 19;
b3) is formed by single-stranded RNA molecules shown in SEQ ID NO: 15, SEQ ID NO: 16, SEQ ID NO: 17, SEQ ID NO: 18 and SEQ ID NO: 22;
b4) is formed by single-stranded RNA molecules shown in SEQ ID NO: 15, SEQ ID NO: 16, SEQ ID NO: 17, SEQ ID NO: 23, SEQ ID NO: 24 and SEQ ID NO: 25;
b5) is formed by single-stranded RNA molecules shown in SEQ ID NO: 15, SEQ ID NO: 16, SEQ ID NO: 17, SEQ ID NO: 20, SEQ ID NO: 23, SEQ ID NO: 24, SEQ ID NO: 26 and SEQ ID NO: 27;
or, the polymeric nucleic acid molecule for simultaneously interfering with expressions of the target genes BIRC5, CTNNB, COPS5 and CLU is formed by single-stranded RNA molecules shown in SEQ ID NO: 28, SEQ ID NO: 29, SEQ ID NO: 30 and SEQ ID NO: 31;
or, the polymeric nucleic acid molecule for simultaneously interfering with expressions of the target genes BIRC5, CTNNB, COPS5, CLU, EIF4E and HIF1A is formed by single-stranded RNA molecules shown in SEQ ID NO: 28, SEQ ID NO: 29, SEQ ID NO: 30, SEQ ID NO: 32, SEQ ID NO: 33 and SEQ ID NO: 34;
or, the polymeric nucleic acid molecule for simultaneously interfering with expressions of the target genes SOD1, PP1B, P65 and VEGFA is the following c1)-c3):
c1) is formed by single-stranded RNA molecules shown in SEQ ID NO: 35, SEQ ID NO: 36, SEQ ID NO: 37 and SEQ ID NO: 38;
c2) is formed by single-stranded RNA molecules shown in SEQ ID NO: 35, SEQ ID NO: 36, SEQ ID NO: 37, SEQ ID NO: 38, SEQ ID NO: 39 and SEQ ID NO: 40;
c3) is formed by single-stranded RNA molecules shown in SEQ ID NO: 35, SEQ ID NO: 36, SEQ ID NO: 37, SEQ ID NO: 38 and SEQ ID NO: 41;
or, the polymeric nucleic acid molecule for interfering with an expression of the target gene VEGFA is the following d1)-d11):
d1) is formed by single-stranded RNA molecules shown in SEQ ID NO: 42, SEQ ID NO: 43, SEQ ID NO: 44, SEQ ID NO: 45, SEQ ID NO:46 and SEQ ID NO: 47;
d2) is formed by single-stranded RNA molecules shown in SEQ ID NO: 48, SEQ ID NO: 49, SEQ ID NO: 50, SEQ ID NO: 51, SEQ ID NO: 52 and SEQ ID NO: 53;
d3) is formed by single-stranded RNA molecules shown in SEQ ID NO: 54, SEQ ID NO: 55, SEQ ID NO: 56, SEQ ID NO: 57, SEQ ID NO: 58 and SEQ ID NO: 59;
d4) is formed by single-stranded RNA molecules shown in SEQ ID NO: 42, SEQ ID NO: 43, SEQ ID NO: 45, SEQ ID NO: 46 and SEQ ID NO: 47;
d5) is formed by single-stranded RNA molecules shown in SEQ ID NO: 48, SEQ ID NO: 49, SEQ ID NO: 51, SEQ ID NO: 52 and SEQ ID NO: 53;
d6) is formed by single-stranded RNA molecules shown in SEQ ID NO: 54, SEQ ID NO: 55, SEQ ID NO: 57, SEQ ID NO: 58 and SEQ ID NO: 59;
d7) is formed by single-stranded RNA molecules shown in SEQ ID NO: 42, SEQ ID NO: 45, SEQ ID NO: 46 and SEQ ID NO: 47;
d8) is formed by single-stranded RNA molecules shown in SEQ ID NO: 48, SEQ ID NO: 51, SEQ ID NO: 52 and SEQ ID NO: 53;
d9) is formed by single-stranded RNA molecules shown in SEQ ID NO: 54, SEQ ID NO: 57, SEQ ID NO: 58 and SEQ ID NO: 59;
d10) is formed by single-stranded RNA molecules shown in SEQ ID NO: 60, SEQ ID NO: 61, SEQ ID NO: 62, SEQ ID NO: 63, SEQ ID NO: 64 and SEQ ID NO: 65;
d11) is formed by single-stranded RNA molecules shown in SEQ ID NO: 66, SEQ ID NO: 67, SEQ ID NO: 68, SEQ ID NO: 69, SEQ ID NO: 70 and SEQ ID NO: 71;
or, the polymeric nucleic acid molecule for interfering with an expression of the target gene TP53 is the following e1 )-e11):
e1) is formed by single-stranded RNA molecules shown in SEQ ID NO: 72, SEQ ID NO: 73, SEQ ID NO: 74, SEQ ID NO: 75, SEQ ID NO: 76 and SEQ ID NO: 77;
e2) is formed by single-stranded RNA molecules shown in SEQ ID NO: 78, SEQ ID NO: 79, SEQ ID NO: 80, SEQ ID NO: 81, SEQ ID NO: 82 and SEQ ID NO: 83;
e3) is formed by single-stranded RNA molecules shown in SEQ ID NO: 84, SEQ ID NO: 85, SEQ ID NO: 86, SEQ ID NO: 87, SEQ ID NO: 88 and SEQ ID NO: 89;
e4) is formed by single-stranded RNA molecules shown in SEQ ID NO: 73, SEQ ID NO: 74, SEQ ID NO: 75, SEQ ID NO: 76 and SEQ ID NO: 77;
e5) is formed by single-stranded RNA molecules shown in SEQ ID NO: 79, SEQ ID NO: 80, SEQ ID NO: 81, SEQ ID NO: 82 and SEQ ID NO: 83;
e6) is formed by single-stranded RNA molecules shown in SEQ ID NO: 54, SEQ ID NO: 55, SEQ ID NO: 57, SEQ ID NO: 58 and SEQ ID NO: 59;
e7) is formed by single-stranded RNA molecules shown in SEQ ID NO: 74, SEQ ID NO: 75, SEQ ID NO: 76 and SEQ ID NO: 77;
e8) is formed by single-stranded RNA molecules shown in SEQ ID NO: 80, SEQ ID NO: 81, SEQ ID NO: 82 and SEQ ID NO: 83;
e9) is formed by single-stranded RNA molecules shown in SEQ ID NO: 86, SEQ ID NO: 87, SEQ ID NO: 88 and SEQ ID NO: 89;
e10) is formed by single-stranded RNA molecules shown in SEQ ID NO: 90, SEQ ID NO: 91, SEQ ID NO: 92, SEQ ID NO: 93, SEQ ID NO: 94 and SEQ ID NO: 95; and
e11) is formed by single-stranded RNA molecules shown in SEQ ID NO: 96, SEQ ID NO: 97, SEQ ID NO: 98, SEQ ID NO: 99, SEQ ID NO: 100 and SEQ ID NO: 101.

19. A derivative of the polymeric nucleic acid molecule as claimed in claim 1 is any one of the following (m1)-(m5):
(m1) the polymeric nucleic acid molecule as claimed in claim 1 is deleted or added one or more nucleotides, to obtain a derivative of the polymeric nucleic acid molecule having the same function as the polymeric nucleic acid molecule;
(m2) the polymeric nucleic acid molecule as claimed in claim 1 is performed nucleotide substitution or modification, to obtain a derivative of the polymeric nucleic acid molecule having the same function as the polymeric nucleic acid molecule;
(m3) a backbone of the polymeric nucleic acid molecule as claimed in claim 1 is transformed into a phosphorothioate backbone, to obtain a derivative of the polymeric nucleic acid molecule having the same function as the polymeric nucleic acid molecule;
(m4) a peptide nucleic acid, a locked nucleic acid or an unlocked nucleic acid coded by the polymeric nucleic acid molecule as claimed in claim 1 is used, to obtain a derivative of the polymeric nucleic acid molecule having the same function as the polymeric nucleic acid molecule; and
(m5) one end or middle of the polymeric nucleic acid molecule as claimed in claim 1 is linked with a signal molecule and/or an active molecule and/or a functional group, to obtain a derivative of the polymeric nucleic acid molecule having the same function as the polymeric nucleic acid molecule.

20. A preparation method for the polymeric nucleic acid molecule as claimed in claim 1, comprising the following steps:
M1) synthesizing the X-type nucleic acid molecule and/or the H-type nucleic acid molecule and/or the C-type nucleic acid molecule of the polymeric nucleic acid molecule as claimed in claim 1; and
M2) annealing the X-type nucleic acid molecule and/or the H-type nucleic acid molecule and/or the C-type nucleic acid molecule, to obtain the polymeric nucleic acid molecule.)

21. An application of the polymeric nucleic acid molecule as claimed in claim 1 in the following method -A1) or A2):
A1) controlling a target gene expression level in a cell;
A2) preparing a product for preventing and/or relieving and/or treating a disease caused by the target gene expression.

22. The application as claimed in claim 21, wherein the controlling is inhibition or reduction or interference;
preferably, the cell is a tumor cell.

23. (canceled)

24. The application as claimed in claim 21, wherein the target gene is a disease-related gene;
or, the disease-related gene is a tumor-related gene;
or, the tumor-related gene is at least one of the following genes: PPIB, p65, BIRC5, CTNNB, COPS5, CLU, EIF4E, HIF1A, TP53, VEGFA and SOD1.

25. A reagent or a kit or a drug for inhibiting or reducing or interfering with a target gene expression level in a cell, comprising the polymeric nucleic acid molecule as claimed in claim 1 .

26. A method for inhibiting or reducing or interfering with a target gene expression level in a cell, comprising the following steps:
the polymeric nucleic acid molecule as claimed in claim 1 is introduced into the cell, and
the expression level of the target gene in the cell is inhibited or reduced.


SEQUENCE LISTING (125 sequences)

SEQ ID NO: 1 - RNA, 30 nt, Artificial Sequence
  Synthesized
  misc_feature (1)..(18): target sequence
  misc_feature (24)..(30): the nucleotide ribose is modified by 2-O-methyl ribose
agaugcucuuuccuccugcaagguguauuu

SEQ ID NO: 2 - RNA, 30 nt, Artificial Sequence
  Synthesized
  misc_feature (1)..(18): target sequence of PPIB
  misc_feature (24)..(30): the nucleotide ribose is modified by 2-O-methyl ribose
aaauacaccuugacgguggaugaagaugua

SEQ ID NO: 3 - RNA, 30 nt, Artificial Sequence
  Synthesized
  misc_feature (1)..(18): target sequence of PPIB
  misc_feature (24)..(30): the nucleotide ribose is modified by 2-O-methyl ribose
uacaucuucaucuccaauucucuucggaaa

SEQ ID NO: 4 - RNA, 30 nt, Artificial Sequence
  Synthesized
  misc_feature (1)..(18): target sequence of PPIB
  misc_feature (24)..(30): the nucleotide ribose is modified by 2-O-methyl ribose
uuuccgaagagaccaaaggaaagagcaucu

SEQ ID NO: 5 - RNA, 30 nt, Artificial Sequence
  Synthesized
  misc_feature (1)..(18): target sequence of PPIB
  misc_feature (24)..(30): the nucleotide ribose is modified by 2-O-methyl ribose
uuuccgaagagaccaaagucuacgagaaag

SEQ ID NO: 6 - RNA, 16 nt, Artificial Sequence
  Synthesized
  misc_feature (10)..(16): the nucleotide ribose is modified by 2-O-methyl ribose
ggaggaaagagcaucu

SEQ ID NO: 7 - RNA, 16 nt, Artificial Sequence
  Synthesized
  misc_feature (1)..(16): the nucleotide ribose is modified by 2-O-methyl ribose
cuuucucguagacuuu

SEQ ID NO: 8 - RNA, 24 nt, Artificial Sequence
  Synthesized
  misc_feature (1)..(24): the nucleotide ribose at positions of 1-4, 7-10, 13-16, 19-22 is modified by 2-O-methyl ribose
cuuuggauuggacaccgucaggag

SEQ ID NO: 9 - RNA, 30 nt, Artificial Sequence
  Synthesized
  misc_feature (1)..(18): target sequence of PPIB
  misc_feature (24)..(30): the nucleotide ribose is modified by 2-O-methyl ribose
agaugcucuuuccuccugcaugaaggugcu

SEQ ID NO: 10 - RNA, 30 nt, Artificial Sequence
  Synthesized
  misc_feature (1)..(18): target sequence of PPIB
  misc_feature (24)..(30): the nucleotide ribose is modified by 2-O-methyl ribose
agcaccuucauguugcgucaagguguauuu

SEQ ID NO: 11 - RNA, 30 nt, Artificial Sequence
  Synthesized
uuuccgaagagaccaaagcguguaaucaag

SEQ ID NO: 12 - RNA, 30 nt, Artificial Sequence
  Synthesized
  misc_feature (1)..(18): target sequence of PPIB
  misc_feature (24)..(30): the nucleotide ribose is modified by 2-O-methyl ribose
cuugauuacacgauggaagaaagagcaucu

SEQ ID NO: 13 - RNA, 30 nt, Artificial Sequence
  Synthesized
  misc_feature (1)..(18): target sequence of PPIB
  misc_feature (24)..(30): target sequence of PPIB
cuugauuacacgauggaaucuacgagaaag

SEQ ID NO: 14 - RNA, 16 nt, Artificial Sequence
  Synthesized
  misc_feature (1)..(16): the nucleotide ribose is modified by 2-O-methyl ribose
cuuucucguagauucc

SEQ ID NO: 15 - RNA, 30 nt, Artificial Sequence
  Synthesized
  misc_feature (1)..(18): target sequence of P65
  misc_feature (24)..(30): the nucleotide ribose is modified by 2-O-methyl ribose
uguguagccauugaucuugcaucaugaaga

SEQ ID NO: 16 - RNA, 30 nt, Artificial Sequence
  Synthesized
  misc_feature (1)..(18): target sequence of P65
  misc_feature (24)..(30): the nucleotide ribose is modified by 2-O-methyl ribose
ucuucaugaugcucuugaauaccaccaaga

SEQ ID NO: 17 - RNA, 30 nt, Artificial Sequence
  Synthesized
  misc_feature (1)..(18): target sequence of P65
  misc_feature (24)..(30): the nucleotide ribose is modified by 2-O-methyl ribose
ucuuggugguaucugugcucgucaccggau

SEQ ID NO: 18 - RNA, 30 nt, Artificial Sequence
  Synthesized
  misc_feature (1)..(18): target sequence of P65
  misc_feature (24)..(30): the nucleotide ribose is modified by 2-O-methyl ribose.
auccggugacgaucgucuaauggcuacaca

SEQ ID NO: 19 - RNA, 30 nt, Artificial Sequence
  Synthesized
  misc_feature (1)..(18): target sequence of P65
  misc_feature (24)..(30): the nucleotide ribose is modified by 2-O-methyl ribose
auccggugacgaucgucuacacaucgguaa

SEQ ID NO: 20 - RNA, 16 nt, Artificial Sequence
  Synthesized
  misc_feature (10)..(16): the nucleotide ribose is modified by 2-O-methyl ribose
gaucaauggcuacaca

SEQ ID NO: 21 - RNA, 16 nt, Artificial Sequence
  Synthesized
  misc_feature (1)..(16): the nucleotide ribose is modified by 2-O-methyl ribose
uuaccgauguguagac

SEQ ID NO: 22 - RNA, 24 nt, Artificial Sequence
  Synthesized
  misc_feature (1)..(24): the nucleotide ribose at positions of 1-4, 7-10, 13-16, 19-22  is modified by 2-O-methyl ribose
agacgagcacagucaagaaagauc

SEQ ID NO: 23 - RNA, 30 nt, Artificial Sequence
  Synthesized
  misc_feature (1)..(18): target sequence of P65
  misc_feature (24)..(30): the nucleotide ribose is modified by 2-O-methyl ribose.
auccggugacgaucgucuucaggagaugaa

SEQ ID NO: 24 - RNA, 30 nt, Artificial Sequence
  Synthesized
  misc_feature (1)..(18): target sequence of P65
  misc_feature (1)..(18): target sequence of P65
  misc_feature (24)..(30): the nucleotide ribose is modified by 2-O-methyl ribose
uucaucuccugaaaggagaucagcuccuaa

SEQ ID NO: 25 - RNA, 30 nt, Artificial Sequence
  Synthesized
  misc_feature (1)..(18): target sequence of P65
  misc_feature (24)..(30): the nucleotide ribose is modified by 2-O-methyl ribose
uuaggagcugaucugacuaauggcuacaca

SEQ ID NO: 26 - RNA, 30 nt, Artificial Sequence
  Synthesized
  misc_feature (1)..(18): target sequence
  misc_feature (24)..(30): the nucleotide ribose is modified by 2-O-methyl ribose
uuaggagcugaucugacuacacaucgguaa

SEQ ID NO: 27 - RNA, 16 nt, Artificial Sequence
  Synthesized
  misc_feature (1)..(16): the nucleotide ribose is modified by 2-O-methyl ribose
uuaccgauguguaguc

SEQ ID NO: 28 - RNA, 30 nt, Artificial Sequence
  Synthesized
  misc_feature (1)..(18): target sequence of BIRC5
  misc_feature (24)..(30): he nucleotide ribose is modified by 2-O-methyl ribose
agaagaaacacugggccaacugcugaucuu

SEQ ID NO: 29 - RNA, 30 nt, Artificial Sequence
  Synthesized
  misc_feature (1)..(18): target sequence of CTNNB1
  misc_feature (24)..(30): the nucleotide ribose is modified by 2-O-methyl ribose
aagaucagcagucucauuuggucaggucuu

SEQ ID NO: 30 - RNA, 30 nt, Artificial Sequence
  Synthesized
  misc_feature (1)..(18): target sequence of COPS5
  misc_feature (24)..(30): the nucleotide ribose is modified by 2-O-methyl ribose
aagaccugaccagugguauuugacucugau

SEQ ID NO: 31 - RNA, 30 nt, Artificial Sequence
  Synthesized
  misc_feature (1)..(18): target sequence of CLU
  misc_feature (24)..(30): the nucleotide ribose is modified by 2-O-methyl ribose
aucagagucaaagagcuuaguguuucuucu

SEQ ID NO: 32 - RNA, 30 nt, Artificial Sequence
  Synthesized
  misc_feature (24)..(30): the nucleotide ribose is modified by 2-O-methyl ribose
ucaaguugcuggucaucaggagguugcuaa

SEQ ID NO: 33 - RNA, 30 nt, Artificial Sequence
  Synthesized
  misc_feature (1)..(18): target sequence of EIF4E
  misc_feature (24)..(30): the nucleotide ribose is modified by 2-O-methyl ribose
uuagcaaccuccugauuaaguguuucuucu

SEQ ID NO: 34 - RNA, 30 nt, Artificial Sequence
  Synthesized
  misc_feature (1)..(18): target sequence of CLU
  misc_feature (24)..(30): the nucleotide ribose is modified by 2-O-methyl ribose
aucagagucaaagagcuuccagcaacuuga

SEQ ID NO: 35 - RNA, 30 nt, Artificial Sequence
  Synthesized
  misc_feature (1)..(18): target sequence of SOD1
  misc_feature (24)..(30): the nucleotide ribose is modified by 2-O-methyl ribose
uacuuucuucauuuccaccuguuccaaaaa

SEQ ID NO: 36 - RNA, 30 nt, Artificial Sequence
  Synthesized
  misc_feature (1)..(18): target sequence of PPIB
  misc_feature (24)..(30): the nucleotide ribose is modified by 2-O-methyl ribose
uuuuuggaacagucuuucugagaccuucaa

SEQ ID NO: 37 - RNA, 30 nt, Artificial Sequence
  Synthesized
  misc_feature (1)..(18): target sequence of P65
  misc_feature (24)..(30): the nucleotide ribose is modified by 2-O-methyl ribose
uugaaggucucauauguccaugcagauuau

SEQ ID NO: 38 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
auaaucugcauggugaugaugaagaaagua

SEQ ID NO: 39 - RNA, 16 nt, Artificial Sequence
  Synthesized
  misc_feature (10)..(16): the nucleotide ribose is modified by 2-O-methyl ribose
ggaaaugaagaaagua

SEQ ID NO: 40 - RNA, 16 nt, Artificial Sequence
  Synthesized
  misc_feature (1)..(16): the nucleotide ribose is modified by 2-O-methyl ribose
uacuuucuucaucauc

SEQ ID NO: 41 - RNA, 24 nt, Artificial Sequence
  Synthesized
  misc_feature (1)..(24): the nucleotide ribose at positions of 1-4, 7-10, 13-16 and 19-22 is modified by 2-O-methyl ribose
caucacgacauagaaagaguggaa

SEQ ID NO: 42 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
agcagaaaguucaugguuucuugggugcau

SEQ ID NO: 43 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
augcacccaagacagcagagaucgaguaca

SEQ ID NO: 44 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
uguacucgaucucaucaggguggacaucuu

SEQ ID NO: 45 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
aagauguccaccagggucaugcggaucaaa

SEQ ID NO: 46 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
uuugauccgcauaaucugggccagcacaua

SEQ ID NO: 47 - RNA, 30 nt, Artificial Sequence
  Synthesized
  misc_feature (1)..(18): target sequence of VEGFA
  misc_feature (24)..(30): the nucleotide ribose is modified by 2-O-methyl ribose
uaugugcuggccuugguggaacuuucugcu

SEQ ID NO: 48 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
agcagaaaguucaugguucuugggugcau

SEQ ID NO: 49 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
augcacccaagacagcaagaucgaguaca

SEQ ID NO: 50 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
uguacucgaucucaucagguggacaucuu

SEQ ID NO: 51 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
aagauguccaccaggguaugcggaucaaa

SEQ ID NO: 52 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
uuugauccgcauaaucuggccagcacaua

SEQ ID NO: 53 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
uaugugcuggccuuggugaacuuucugcu

SEQ ID NO: 54 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
agcagaaaguucaugucuugggugcau

SEQ ID NO: 55 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
augcacccaagacagagaucgaguaca

SEQ ID NO: 56 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
uguacucgaucucaugguggacaucuu

SEQ ID NO: 57 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
aagauguccaccaggaugcggaucaaa

SEQ ID NO: 58 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
uuugauccgcauaauggccagcacaua

SEQ ID NO: 59 - RNA, 27 nt, Artificial Sequence
  Synthesized
  misc_feature (1)..(15): target sequence of VEGFA
  misc_feature (21)..(27): the nucleotide ribose is modified by 2-O-methyl ribose
uaugugcuggccuuggaacuuucugcu

SEQ ID NO: 60 - RNA, 28 nt, Artificial Sequence
  Synthesized
  misc_feature (1)..(17): target sequence of VEGFA
  misc_feature (22)..(28): the nucleotide ribose is modified by 2-O-methyl ribose.
agcagaaaguucauggucuugggugcau

SEQ ID NO: 61 - RNA, 28 nt, Artificial Sequence
  Synthesized
  misc_feature (1)..(17): target sequence of VEGFA
  misc_feature (22)..(28): the nucleotide ribose is modified by 2-O-methyl ribose
augcacccaagacagcagaucgaguaca

SEQ ID NO: 62 - RNA, 28 nt, Artificial Sequence
  Synthesized
  misc_feature (1)..(17): target sequence of VEGFA
  misc_feature (22)..(28): the nucleotide ribose is modified by 2-O-methyl ribose
uguacucgaucucaucaguggacaucuu

SEQ ID NO: 63 - RNA, 28 nt, Artificial Sequence
  Synthesized
  misc_feature (1)..(17): target sequence of VEGFA
  misc_feature (22)..(28): the nucleotide ribose is modified by 2-O-methyl ribose
aagauguccaccaggguugcggaucaaa

SEQ ID NO: 64 - RNA, 28 nt, Artificial Sequence
  Synthesized
  misc_feature (1)..(17): target sequence of VEGFA
  misc_feature (22)..(28): the nucleotide ribose is modified by 2-O-methyl ribose
uuugauccgcauaaucugccagcacaua

SEQ ID NO: 65 - RNA, 28 nt, Artificial Sequence
  Synthesized
  misc_feature (1)..(17): target sequence of VEGFA
  misc_feature (22)..(28): the nucleotide ribose is modified by 2-O-methyl ribose
uaugugcuggccuugguaacuuucugcu

SEQ ID NO: 66 - RNA, 26 nt, Artificial Sequence
  Synthesized
  misc_feature (1)..(16): target sequence of VEGFA
  misc_feature (20)..(26): the nucleotide ribose is modified by 2-O-methyl ribose
agcagaaaguucaugguugggugcau

SEQ ID NO: 67 - RNA, 26 nt, Artificial Sequence
  Synthesized
  misc_feature (1)..(16): target sequence of VEGFA
  misc_feature (20)..(26): the nucleotide ribose is modified by 2-O-methyl ribose
augcacccaagacagcaucgaguaca

SEQ ID NO: 68 - RNA, 26 nt, Artificial Sequence
  Synthesized
  misc_feature (1)..(16): target sequence of VEGFA
  misc_feature (20)..(26): the nucleotide ribose is modified by 2-O-methyl ribose
uguacucgaucucaucuggacaucuu

SEQ ID NO: 69 - RNA, 26 nt, Artificial Sequence
  Synthesized
  misc_feature (1)..(16): target sequence of VEGFA
  misc_feature (20)..(26): the nucleotide ribose is modified by 2-O-methyl ribose
aagauguccaccaggggcggaucaaa

SEQ ID NO: 70 - RNA, 26 nt, Artificial Sequence
  Synthesized
  misc_feature (1)..(16): target sequence of VEGFA
  misc_feature (20)..(26): the nucleotide ribose is modified by 2-O-methyl ribose.
uuugauccgcauaaucccagcacaua

SEQ ID NO: 71 - RNA, 26 nt, Artificial Sequence
  Synthesized
  misc_feature (1)..(16): target sequence of VEGFA
  misc_feature (20)..(26): the nucleotide ribose is modified by 2-O-methyl ribose
uaugugcuggccuuggacuuucugcu

SEQ ID NO: 72 - RNA, 30 nt, Artificial Sequence
  Synthesized
  misc_feature (1)..(18): target sequence of TP53
  misc_feature (24)..(30): the nucleotide ribose is modified by 2-O-methyl ribose.
uguggaaucaacccacaguuugcgugugga

SEQ ID NO: 73 - RNA, 30 nt, Artificial Sequence
  Synthesized
  misc_feature (1)..(18): target sequence of TP53
  misc_feature (24)..(30): the nucleotide ribose is modified by 2-O-methyl ribose
uccacacgcaaauuuccuacagaaacacuu

SEQ ID NO: 74 - RNA, 30 nt, Artificial Sequence
  Synthesized
  misc_feature (1)..(18): target sequence of TP53
  misc_feature (24)..(30): the nucleotide ribose is modified by 2-O-methyl ribose
aaguguuucugucauccaacuacaugugua

SEQ ID NO: 75 - RNA, 30 nt, Artificial Sequence
  Synthesized
  misc_feature (1)..(18): target sequence of TP53
  misc_feature (24)..(30): the nucleotide ribose is modified by 2-O-methyl ribose
uacacauguaguuguaguugguaaucuacu

SEQ ID NO: 76 - RNA, 30 nt, Artificial Sequence
  Synthesized
  misc_feature (1)..(18): target sequence of TP53
  misc_feature (24)..(30): the nucleotide ribose is modified by 2-O-methyl ribose
aguagauuaccacuggagucuccgcaagaa

SEQ ID NO: 77 - RNA, 30 nt, Artificial Sequence
  Synthesized
  misc_feature (1)..(18): target sequence of VEGFA
  misc_feature (24)..(30): the nucleotide ribose is modified by 2-O-methyl ribose.
uucuugcggagauucucuguugauuccaca

SEQ ID NO: 78 - RNA, 29 nt, Artificial Sequence
  Synthesized
  misc_feature (1)..(17): target sequence of TP53
  misc_feature (23)..(29): the nucleotide ribose is modified by 2-O-methyl ribose
uguggaaucaacccacauuugcgugugga

SEQ ID NO: 79 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
uccacacgcaaauuuccacagaaacacuu

SEQ ID NO: 80 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
aaguguuucugucauccacuacaugugua

SEQ ID NO: 81 - RNA, 29 nt, Artificial Sequence
  Synthesized
  misc_feature (1)..(17): target sequence of VEGFA
  misc_feature (23)..(29): the nucleotide ribose is modified by 2-O-methyl ribose
uacacauguaguuguagugguaaucuacu

SEQ ID NO: 82 - RNA, 29 nt, Artificial Sequence
  Synthesized
  misc_feature (1)..(17): target sequence of TP53
  misc_feature (23)..(29): the nucleotide ribose is modified by 2-O-methyl ribose
aguagauuaccacuggaucuccgcaagaa

SEQ ID NO: 83 - RNA, 29 nt, Artificial Sequence
  Synthesized
  misc_feature (1)..(17): target sequence of TP53
  misc_feature (23)..(29): the nucleotide ribose is modified by 2-O-methyl ribose
uucuugcggagauucucguugauuccaca

SEQ ID NO: 84 - RNA, 27 nt, Artificial Sequence
  Synthesized
  misc_feature (1)..(15): target sequence of TP53
  misc_feature (21)..(27): the nucleotide ribose is modified by 2-O-methyl ribose
uguggaaucaacccauuugcgugugga

SEQ ID NO: 85 - RNA, 27 nt, Artificial Sequence
  Synthesized
  misc_feature (1)..(15): target sequence of TP53
  misc_feature (21)..(27): the nucleotide ribose is modified by 2-O-methyl ribose
uccacacgcaaauuuacagaaacacuu

SEQ ID NO: 86 - RNA, 27 nt, Artificial Sequence
  Synthesized
  misc_feature (1)..(15): target sequence of TP53
  misc_feature (21)..(27): the nucleotide ribose is modified by 2-O-methyl ribose
aaguguuucugucauacuacaugugua

SEQ ID NO: 87 - RNA, 27 nt, Artificial Sequence
  Synthesized
  misc_feature (1)..(15): target sequence of TP53
  misc_feature (21)..(27): the nucleotide ribose is modified by 2-O-methyl ribose
uacacauguaguuguugguaaucuacu

SEQ ID NO: 88 - RNA, 27 nt, Artificial Sequence
  Synthesized
  misc_feature (1)..(15): target sequence of TP53
  misc_feature (21)..(27): the nucleotide ribose is modified by 2-O-methyl ribose.
aguagauuaccacugucuccgcaagaa

SEQ ID NO: 89 - RNA, 27 nt, Artificial Sequence
  Synthesized
  misc_feature (1)..(15): target sequence of TP53
  misc_feature (21)..(27): the nucleotide ribose is modified by 2-O-methyl ribose.
uucuugcggagauucguugauuccaca

SEQ ID NO: 90 - RNA, 28 nt, Artificial Sequence
  Synthesized
  misc_feature (1)..(17): target sequence of TP53
  misc_feature (22)..(28): the nucleotide ribose is modified by 2-O-methyl ribose
uguggaaucaacccacauugcgugugga

SEQ ID NO: 91 - RNA, 28 nt, Artificial Sequence
  Synthesized
  misc_feature (1)..(17): target sequence of TP53
  misc_feature (22)..(28): the nucleotide ribose is modified by 2-O-methyl ribose
uccacacgcaaauuucccagaaacacuu

SEQ ID NO: 92 - RNA, 28 nt, Artificial Sequence
  Synthesized
  misc_feature (1)..(17): target sequence of TP53
  misc_feature (22)..(28): the nucleotide ribose is modified by 2-O-methyl ribose
aaguguuucugucaucccuacaugugua

SEQ ID NO: 93 - RNA, 28 nt, Artificial Sequence
  Synthesized
  misc_feature (1)..(17): target sequence of TP53
  misc_feature (22)..(28): the nucleotide ribose is modified by 2-O-methyl ribose
uacacauguaguuguaggguaaucuacu

SEQ ID NO: 94 - RNA, 28 nt, Artificial Sequence
  Synthesized
  misc_feature (1)..(17): target sequence of TP53
  misc_feature (22)..(28): the nucleotide ribose is modified by 2-O-methyl ribose
aguagauuaccacuggacuccgcaagaa

SEQ ID NO: 95 - RNA, 28 nt, Artificial Sequence
  Synthesized
  misc_feature (1)..(17): target sequence of TP53
  misc_feature (22)..(28): the nucleotide ribose is modified by 2-O-methyl ribose
uucuugcggagauucucuugauuccaca

SEQ ID NO: 96 - RNA, 26 nt, Artificial Sequence
  Synthesized
  misc_feature (1)..(15): target sequence of TP53
  misc_feature (20)..(26): the nucleotide ribose is modified by 2-O-methyl ribose.
uguggaaucaacccacugcgugugga

SEQ ID NO: 97 - RNA, 26 nt, Artificial Sequence
  Synthesized
  misc_feature (1)..(15): target sequence of TP53
  misc_feature (20)..(26): the nucleotide ribose is modified by 2-O-methyl ribose
uccacacgcaaauuucagaaacacuu

SEQ ID NO: 98 - RNA, 26 nt, Artificial Sequence
  Synthesized
  misc_feature (1)..(15): target sequence of TP53
  misc_feature (20)..(26): the nucleotide ribose is modified by 2-O-methyl ribose
aaguguuucugucaucuacaugugua

SEQ ID NO: 99 - RNA, 26 nt, Artificial Sequence
  Synthesized
  misc_feature (1)..(15): target sequence of TP53
  misc_feature (20)..(26): the nucleotide ribose is modified by 2-O-methyl ribose
uacacauguaguuguaguaaucuacu

SEQ ID NO: 100 - RNA, 26 nt, Artificial Sequence
  Synthesized
  misc_feature (1)..(15): target sequence of TP53
  misc_feature (20)..(26): the nucleotide ribose is modified by 2-O-methyl ribose
aguagauuaccacugguccgcaagaa

SEQ ID NO: 101 - RNA, 25 nt, Artificial Sequence
  Synthesized
  misc_feature (1)..(15): target sequence of TP53
  misc_feature (19)..(25): the nucleotide ribose is modified by 2′-O-methyl ribose
ucuugcggagauucuugauuccaca

SEQ ID NO: 102 - DNA, 21 bp, Artificial Sequence
  a synthesized single-strand of a negative control group siRNA
uucuccgaacgugucacgutt

SEQ ID NO: 103 - DNA, 21 bp, Artificial Sequence
  a synthesized single-strand of a negative control group siRNA
acgugacacguucggagaatt

SEQ ID NO: 104 - DNA, 20 bp, Artificial Sequence
  Synthesized
ttgtgcctgtcctgggagag

SEQ ID NO: 105 - DNA, 21 bp, Artificial Sequence
  Synthesized
ggagaggagctggtgttgttg

SEQ ID NO: 106 - DNA, 20 bp, Artificial Sequence
  Synthesized
gccctaacgtgttatctgtc

SEQ ID NO: 107 - DNA, 20 bp, Homo sapiens
cgctttctctgagcattctg

SEQ ID NO: 108 - DNA, 22 bp, Homo sapiens
ggaggttgctaacccagaacac

SEQ ID NO: 109 - DNA, 20 bp, Homo sapiens
ggagatcagccgcaggtttg

SEQ ID NO: 110 - DNA, 21 bp, Homo sapiens
gagggcagaatcatcacgaag

SEQ ID NO: 111 - DNA, 22 bp, Homo sapiens
actcgatctcatcagggtactc

SEQ ID NO: 112 - DNA, 20 bp, Homo sapiens
ggcaagcatgtggtgtttgg

SEQ ID NO: 113 - DNA, 21 bp, Homo sapiens
ggtttatcccggctgtctgtc

SEQ ID NO: 114 - DNA, 20 bp, Homo sapiens
gggaaggaacgctgtcagag

SEQ ID NO: 115 - DNA, 21 bp, Homo sapiens
tagcctcagggtactccatca

SEQ ID NO: 116 - DNA, 20 bp, Homo sapiens
gcagggcatcatcaatttcg

SEQ ID NO: 117 - DNA, 20 bp, Homo sapiens
gaatccatgcaggccttcag

SEQ ID NO: 118 - DNA, 20 bp, Homo sapiens
agaactggcccttcttggag

SEQ ID NO: 119 - DNA, 20 bp, Homo sapiens
gaaacactgggccaagtctg

SEQ ID NO: 120 - DNA, 19 bp, Homo sapiens
gctcgggatgttcacaacc

SEQ ID NO: 121 - DNA, 19 bp, Homo sapiens
ccctgcagctactctttgg

SEQ ID NO: 122 - DNA, 24 bp, Homo sapiens
tggaataaatactgggtgaatacg

SEQ ID NO: 123 - DNA, 19 bp, Homo sapiens
ggcttctgactgctctaac

SEQ ID NO: 124 - DNA, 22 bp, Homo sapiens
caaggcgaagaccagtactatc

SEQ ID NO: 125 - DNA, 19 bp, Homo sapiens
cagtgacaccggaaggaac